(19) United States
(12) Patent Application Publication
(10) Pub. No.: US 2020/0237581 A1
(43) Pub. Date: Jul. 30, 2020

(54) ABSORBENT ARTICLES HAVING ABSORBENT LAMINATES AND HIGH-BULK, LOW SHEAR TOPSHEETS

(71) Applicant: ATTENDS HEALTHCARE PRODUCTS, INC., Raleigh, NC (US)

(72) Inventors: Harry J. CHMIELEWSKI, Wake Forest, NC (US); Tom MORSE, Raleigh, NC (US); Edward L. SEAMES, Durham, NC (US)

(73) Assignee: ATTENDS HEALTHCARE PRODUCTS, INC., Raleigh, NC (US)

(21) Appl. No.: 16/774,687

(22) Filed: Jan. 28, 2020

Related U.S. Application Data
(60) Provisional application No. 62/797,653, filed on Jan. 28, 2019.

(51) Int. Cl. A61F 13/537

(52) U.S. Cl. CPC A61F 13/53747; A61F 2013/530591

(57) ABSTRACT

An absorbent article includes a liquid-permeable nonwoven topsheet having a bulk greater than or equal to 10 cm³/g, a backsheet, and one or more absorbent laminates disposed between the topsheet and the backsheet. Each of the laminate(s) can comprise one or more substrate laminae, each comprising a nonwoven or tissue, and one or more absorbent laminae, each comprising SAP particles.

### CROSS-REFERENCES WITH RELATED APPLICATIONS

This application claims priority to and the benefit of U.S. Provisional Application No. 62/797,653, filed Jan. 28, 2019, the contents of which is incorporated herein in its entirety.

### FIELD OF INVENTION

The present invention relates generally to absorbent articles and, more particularly, to absorbent articles incorporating resilient absorbent laminates and high-bulk topsheets.

### BACKGROUND

Disposable absorbent products have met with widespread acceptance in the marketplace for a variety of applications, including infant and adult incontinence care and use with non-ambulatory persons, in view of the manner in which such products can provide effective and convenient liquid absorption and retention while maintaining the comfort of the wearer. Disposable absorbent products can include articles that are not worn by a wearer, such as underpads or bed pads that a user can sit or lay on, and articles that are wearable by a user, such as baby diapers, training pants, and adult incontinence briefs and underwear, all of which may be made in disposable forms such as, for example, utilizing nonwoven materials. The terms “absorbent article” and “absorbent garment” refer to garments or articles that absorb and contain exudates and, more specifically, refer to garments or articles that are placed against or in proximity to the body of the wearer to absorb and contain the various exudates such as urine or feces discharged from the body. These garments or articles include diapers, training pants, feminine hygiene products, bibs, wound dressing, bed pads, and adult incontinence products.

Such disposable absorbent articles often include a liquid-pervious topsheet that is configured to be closest to the wearer during use, a liquid-impermeable backsheet or outer cover, and an absorbent core between the topsheet and the backsheet. In some instances, such disposable absorbent articles also include an acquisition-distribution layer (“ADL”) disposed between the topsheet and the absorbent core. “Absorbent core” means a structure positioned between a topsheet and backsheet of an absorbent article for absorbing and containing liquid received by the absorbent article and may comprise one or more substrates, absorbent polymer material, adhesives or other materials to bind absorbent materials in the core.

U.S. Pat. No. 9,398,986 discloses certain prior art examples of training pants, and U.S. Pat. Nos. 6,976,978 and 4,940,464 disclose certain prior art examples of disposable incontinence garments or training pants.

One example of such a disposable absorbent article is shown in FIGS. 1A-1B, which are lower plan and perspective views, respectively, of adult protective underwear 10. Underwear 10 includes a chassis 14 having a front waist portion 18, an opposing rear waist portion 22, and a crotch portion 26 extending longitudinally between front and rear waist portions 18, 22. Chassis 14 further includes an outer surface 30 configured to face away from a wearer during use of the underwear, and an opposing body facing surface 34 configured to face a wearer during use of the underwear.

As shown in FIGS. 1A and 1B, underwear 10 further includes a pair of front elastic side panels 38 and a pair of rear elastic side panels 42 configured to couple rear waist portion 22 to front waist portion 18 in a well-known configuration in which a left side 46 of the chassis defines a first leg opening 50 for a wearer's left leg, and in which a right side 54 of the chassis defines a second leg opening 58 for the wearer's right leg. In the depicted configuration, each of side panels 38, 42 includes a connection portion 62 configured to be coupled to a connection portion 62 of another of side panels 38, 42. Specifically, connection portion 62 of the left one of front side panels 38 is configured to be coupled to connection portion 62 of the left one of rear side panels 42, and connection portion 62 of the right one of front side panels 38 is configured to be coupled to connection portion 62 of the right one of rear side panels 42, such that the waist portions 18, 22 and side panels, 38, 42 cooperate to define a waist opening 66 as shown in FIG. 1B. Connection portions 62 of the respective side panels can be permanently coupled together to define a tear-able side seam 70, such as, for example, via adhesive, ultrasonic, or thermal bonds. “Bonded” refers to the joining, adhering, connecting, attaching, or the like, of two elements. Two elements will be considered to be bonded together when they are bonded directly to one another or indirectly to one another, such as when each is directly bonded to intermediate elements. Such tear-able side seams generally cannot be refastened, and thereby render an article unusable once opened. Alternatively, connection portions 62 of the respective side panels can be removably coupled to define a refastenable or adjustable side seam, such as, for example, via hook-and-loop fasteners. Hook and loop fasteners are mechanical fasteners that include hooks, such as in a hook fastener portion, that are configured to engage loops in a loop fastener portion or in fibers of a sheet of fabric; for example, a nonwoven or woven fabric with fibers that define open or loop-like regions into which the hooks can extend and engage. Examples of such hook and loop fasteners may be referred to as VELCRO.

In a disposable article of the type shown in FIGS. 1A and 1B, outer surface 30 is typically defined by a water-impermeable backsheet, and body facing surface 34 is typically defined by a water-permeable topsheet. As shown in FIG. 1A, underwear 10 also includes an absorbent core 74 and an acquisition-distribution layer or “ADL” 78 disposed between the absorbent core 74 and the topsheet. “Layer” when used in the singular can be a single element or a plurality of elements. For example, a plurality of sheets may together define a single layer, such as, for example, a layer with a particular function to which the sheets of the layer contribute.

An important component of disposable absorbent articles is the absorbent core. Conventional absorbent cores often include cellulosic fluff pulp and superabsorbent polymer or “SAP,” such as hydrogel-forming polymer material. The inclusion of SAP in an absorbent core typically also increases the ability of the absorbent core, relative to a similarly sized absorbent core of fluff pulp alone, to retain absorbed liquid against pressure, thus providing lower rewet and better skin dryness.

Over time, absorbent cores used in such articles have become increasingly thinner with SAP being included in ever-increasing amounts in place of traditional cellulosic pulp and other fillers and absorbents. The benefits of increasing the amount of SAP and decreasing the amount of fluff pulp is that a core can be made thinner while still being able to acquire and store large quantities of discharged body fluids. Such thinner core materials can be made off-line and can be introduced as a continuous web during conventional manufacturing processes. However, such thinner core designs may also have certain technical challenges or issues that often must also be addressed. One such issue is that they typically lack sufficient thickness to provide needed void volume to capture and control gushes of liquid, and typically therefore require an additional acquisition-distribution layer or “ADL” to handle fluid during insults. Another such issue is that thinner absorbent cores with predominantly SAP can have relatively low structural integrity after absorption of liquid. In some instances, relatively small quantities of thermoplastic adhesive material, such as fibrous thermoplastic adhesive material, may be included to physically stabilize the SAP. However, when the basis weight of such an absorbent core is sufficient to provide the desired absorbent performance, the relative stiffness of the absorbent core may be higher and less garment-like, particularly when adhesive or other means is included to physically stabilize the SAP. There exists an inherent conflict between relative thinness, sufficient absorbency, and wet integrity. For example, a light-strike adult incontinence product may need a core that is 25 mm wide to fit comfortably, but may as a result of the relatively small dimension need about 400 gsm of SAP to provide a desired absorbent capacity; however, such a narrow core with such a high basis weight may be difficult to provide in a stable long-running package that is suitable for commercial manufacturing lines.

While these thinner, primarily SAP-containing cores provide advantages, such as, generally offering a better fit to the wearer, they also present various challenges. One such challenge relates to the acquisition and distribution of liquid insults. In conventional core designs the liquid spreads radially from the point where it strikes, or insults, the core. Thus, rather than being dispersed across the core surface generally, its transport may be localized. This challenge is exacerbated by “gel blocking,” which refers to the blocking of liquid transport through the core by the swelling and gelling of the superabsorbent material as it absorbs and retains liquid. Gel blocking may lead to leakage from the article when the core does not have the ability to absorb and retain liquid at a rate that meets or exceeds the rate at which the liquid reaches the core. To address this challenge, the properties of permeability, particularly under the applied pressure of the wearer, and capillarity are often separated between two separate structures. Conventionally, the ADL provides permeability to acquire the liquid as rapidly as it is added to the product (to prevent uncontrolled surface runoff) and then spreads or distributes the acquired liquid over a larger surface area of the absorbent core at a rate that the core can absorb the liquid without undesired internal runoff and leakage. The absorbent core, in turn, wicks the liquid into the core and provides capillary suction to reduce wetness in the ADL, to present a relatively dry ADL surface to the wearer and partially restoring the ADL to its initial state to acquire subsequent insults of liquid.

Examples of certain absorbent cores and articles that address some or all of the foregoing issues are disclosed in U.S. Patent Application Publication No. US 2015/0245958 A1, which is incorporated by reference in its entirety. This application discloses examples of absorbent laminates and folded multi-layer absorbent cores that include superabsorbent polymer particles (“SAP”) and one or more layers of material such as, for example, tissue.

Disposable absorbent articles are sometimes used by non-ambulatory persons, such as those confined to beds, wheelchairs, surgical tables, and/or surgical accessory frames. Non-ambulatory persons often develop decubitus ulcers, frequently referred to as pressure ulcers or bed sores. Pressure ulcers can form on parts of the body where blood circulation and/or the lymphatic system are restricted due to elevated pressure, for example, at the interface of the person's skin and a supporting surface. The pressure at this interface may be referred to in the art as the “transdermal interface pressure” or “TIP.” Pressure ulcers typically develop when TIP or a combination of TIP and shear forces at a given place increases to a level that is high enough to restrict blood flow to soft tissue and waste removal through the lymphatic system, and may occur most often in areas of soft tissue such as skin overlying larger bones that are relatively closer to the skin, for example, the sacrum, coccyx, heels, and hips. Such increases in TIP and shear forces typically occur in soft tissue over such bones because the bone and overlying soft tissue often protrudes beyond surrounding areas of soft tissue and thus supports a relatively larger portion of the person's weight than immediately surrounding areas of soft tissue, and/or because the relatively thinner soft tissue between the bone and the skin is less effective than surrounding areas at distributing the pressure resulting from the weight of the patient. One or both of these factors can contribute to cause TIP to increase and reduce blood and lymphatic system flow to such localized areas. For example, when a person lies on his or her back, the relatively small area of soft tissue overlying the person's heel protrudes farther than surrounding areas of tissue, such as the person's ankle, and is covered by a thinner layer of soft tissue than other parts of the person's leg, for example, the calf. As a result, the person's heel carries more of the weight of the patient's leg, over a smaller area, than surrounding areas of tissue, causing TIP under the person's heel to be higher than surrounding areas of soft tissue and potentially inhibits blood flow to the soft tissue of the person's heel.

A number of attempts have been made in the prior art to reduce the occurrence of pressure ulcers. Conventionally, patient support structures for reducing pressure ulcers can typically be considered either dynamic or static. Dynamic support structures, for example dynamically controllable pads or cushions, are those in which the properties of a support structure are dynamically varied, typically either proactively in various locations to reduce the likelihood of TIP restricting blood flow at any one point for a sufficient period of time to cause a pressure ulcer, or with sensors that indicate TIP and a feedback loop that allows a control system to respond to and reduce localized increases in TIP before a pressure ulcer arises. Although such systems may be practical for patients that are confined to a bed, they may be undesirable for patients in wheelchairs due to the difficulty in mobilizing the equipment associated with such a dynamic support structure. Dynamic support structures, such as cushions, may also be cost prohibitive for some patients due to their relatively high cost.

Static support structures, for example static pads or cushions, generally come in one of two types: (1) bladder-type cushions and (2) foam cushions. Bladder type cushions are typically flexible-walled bags, such as plastic or polymer bags, that are filled with a fluid, such as air or water, or a gel, such as ethylene glycol, polyethylene glycol, silicone, and/or the like. In contrast, foam cushions are typically solid pieces of foam or a foam laminate structure. Foam cushions are generally the less-expensive of these two types of cushion. However, foam cushions may be undesirable in that they may retain thermal energy and result in elevated temperatures at the transdermal interface between the patient's skin and the cushion, which is believed by some to be a contributing factor to the occurrence of decubitus ulcers. In contrast, bladder-type cushions tend to be more expensive, but also tend to distribute force better than foam cushions, thereby reducing TIP which is considered by many in the art to be a primary contributor of pressure ulcers. Some bladder-type cushions may be low in weight and/or density like foam cushions. However, bladder-type cushions, like foam cushions, may tend to retain thermal energy and result in elevated temperatures at the transdermal interface between the patient's skin and the cushion. Another drawback with bladder-type cushions is that they tend to elevate the patient to a height greater than foam cushions.

The use of wearable and non-wearable absorbent articles by such non-ambulatory persons can pose additional challenges. When used, portions of an absorbent article can be disposed between the user and a supporting surface, which can affect TIP. For example, the presence of an absorbent pad between a user and a support surface can increase TIP by 20-25%. The supporting surface may also occlude the outer surface of the absorbent article, which can restrict the diffusion of air and water vapor through the article's outer surface, reducing breathability and thus user comfort. User movement can also result in shear between the user's skin and the absorbent article, which may cause skin irritation and increase the occurrence of bed sores. Skin irritation may also be caused by moisture that remains at the user-facing surface of the article.

Some articles have attempted to address challenges related to TIP. One example of a prior art attempt to incorporate cushions into an incontinence article is disclosed in U.S. Pat. No. 4,114,621, which describes a incontinence garment for a bed-confined patient that includes “somewhat stiff pads” that overlie the patient's hips to distribute force over a greater area of the patient's soft tissue to reduce pressure ulcers. However, these attempts to address challenges related to TIP may be unsatisfactory and may not adequately address the need for articles that can quickly reduce surface moisture after liquid insults and maintain breathability despite being positioned between the user and a non-breathable support surface.

### SUMMARY

Some absorbent articles of the present disclosure address the need in the art for TIP-reducing, breathable, and moisture-wicking articles by incorporating a high-bulk nonwoven topsheet (e.g., having a bulk greater than or equal to 10 cm³/g) in combination with an absorbent core that comprises one or more absorbent laminates. The high-bulk nonwoven topsheet and laminate(s) can interact to reduce TIP relative to conventional articles, and can facilitate rapid moisture transfer from the topsheet to the laminate(s) to promote skin dryness without the need for an ADL. The combination of the high-bulk nonwoven topsheet with the laminate(s) can also facilitate lateral air permeability and thus improved breathability over conventional absorbent articles, even when the outer surface of the article is occluded by a non-breathable support surface.

The topsheet of some articles can include first and second topsheet layers, where the first topsheet layer is configured to slide relative to the second topsheet layer within a slidable region of the topsheet. The coefficient of friction between the topsheet layers can be lower than that between a user's skin and the first topsheet layer. As such, user movement may tend to cause relative sliding between the topsheet layers instead of shear between the user's skin and the first topsheet layer, thereby mitigating skin irritation and the occurrence of bed sores.

Some articles comprise a liquid-permeable nonwoven topsheet, a backsheet, and/or an absorbent core disposed between the topsheet and the backsheet. The nonwoven topsheet, in some articles, has a bulk greater than or equal to 10 cubic centimeters per gram (cm³/g), a basis weight greater than or equal to 50 grams per square meter (gsm), and/or a thickness greater than or equal to 0.50 millimeters (mm). In some articles, a basis weight of the topsheet is between 10 and 120 gsm, optionally between 10 and 65 gsm or between 65 and 120 gsm. The backsheet, in some articles, comprises a nonwoven and, optionally, does not comprise a liquid-impermeable film. In some articles, the backsheet is liquid-impermeable.

The absorbent core, in some articles, is one or more absorbent laminates, optionally two or more laminates, disposed between the topsheet and the backsheet. In some articles, if the one or more laminates comprise two or more laminates, a first one of the laminates is disposed on a second one of the laminates. Each of the laminate(s), in some articles, comprises one or more absorbent laminae, optionally two or more absorbent laminae, and one or more substrate laminae, optionally three or more substrate laminae. Each of the absorbent lamina(e), in some articles, comprises superabsorbent polymer (SAP) particles, which optionally have a basis weight between 20 and 130 gsm, and an adhesive. Each of the substrate lamina(e), in some articles, comprises a nonwoven or a tissue.

A first one of the substrate lamina(e), in some articles, comprises a nonwoven that, optionally, comprises viscose fibers and/or polyethylene terephthalate (PET) fibers and/or has a basis weight between 20 and 40 gsm. In some articles, the first substrate lamina is disposed between first and second ones of the absorbent laminae, the first absorbent lamina is disposed between the first substrate lamina and a second one of the substrate laminae, and/or the second absorbent lamina is disposed between the first substrate lamina and a third one of the substrate laminae. In some articles, for at least one of the laminate(s), the second substrate lamina is disposed on the topsheet such that the second substrate lamina is disposed closer to the topsheet than is the third substrate lamina. At least one of the second and third substrate laminae, in some articles, comprises a tissue. In some articles, the second substrate lamina comprises a nonwoven and the third substrate lamina comprises a tissue. In other articles, the second substrate lamina comprises a tissue and the third substrate lamina comprises a nonwoven. For each of the second and third substrate laminae, in some articles, if the substrate lamina comprises a nonwoven, the nonwoven has basis weight between 40 and 60 gsm.

In some articles, the topsheet comprises first and second topsheet layers and has slidable region that overlies at least a majority of the absorbent core. Each of the first and second topsheet layers, in some articles, comprises a nonwoven and, optionally, at least a portion of the first topsheet layer is gathered within the slidable region. In some articles, an adhesive bonds the first topsheet layer to the second topsheet layer and is disposed outside of the slidable region such that a portion of the first topsheet layer is slidable relative to the second topsheet layer within the slidable region. In some articles, the coefficient of friction between the first and second topsheet layers is lower than a coefficient of friction between the first topsheet layer and the skin of a wearer.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified—and includes what is specified; e.g., substantially 90 degrees includes 90 degrees and substantially parallel includes parallel—as understood by a person of ordinary skill in the art. In any disclosed embodiment, the term “substantially” may be substituted with “within [a percentage] of” what is specified, where the percentage includes 0.1, 1, 5, and 10 percent.

The terms “comprise” and any form thereof such as “comprises” and “comprising,” “have” and any form thereof such as “has” and “having,” and “include” and any form thereof such as “includes” and “including” are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

The term “nonwoven,” as used herein, and per an INDA definition, refers to sheet or web structures bonded together by entangling fiber or filaments, and/or by perforating films, mechanically, thermally, or chemically. They are flat, porous sheets that are made directly from separate fibers or from molten plastic or plastic film. They are not made by weaving or knitting and do not require converting the fibers to yarn. The basis weight of nonwoven fabrics can be expressed as gsm or grams per square meter and as osy or ounces per square yard. The term “film” refers to a membrane-like layer of material formed of one or more polymers, which does not have a form consisting predominately of a web-like structure of fibers and/or other fibers.

The term “liquid impermeable,” when used in describing a material, means that a liquid, such as urine, will not pass through the material, under ordinary use conditions, in a direction generally perpendicular to the plane of the material at the point of liquid contact. The term “breathable,” when used in describing a material, means that the material has a water vapor transmission rate (“WVTR”) of at least about 300 grams/m²/24 hours. “Breathable” materials can be substantially liquid impermeable.

The term “superabsorbent,” “superabsorbent material,” “superabsorbent polymer,” or “SAP” refers to a water-swellable, water-insoluble organic or inorganic material capable, under the most favorable conditions, of absorbing at least about 15 times its weight in an aqueous solution containing 0.9 weight percent sodium chloride and, more desirably, at least about 30 times its weight in an aqueous solution containing 0.9 weight percent sodium chloride and, even more desirably, at least about 50 times its weight in an aqueous solution containing 0.9 weight percent sodium chloride. The SAP materials used in the present methods and articles can be natural, synthetic and modified natural polymers and materials. In addition, the SAP materials can be or include organic compounds such as cross linked polymers. “Cross-linked” is a commonly understood term and refers to any approach for effectively rendering normally water-soluble materials substantially water insoluble, but swellable. Such polymers can include, for example, carboxymethylcellulose, alkali metal salts of polyacrylic acids, polyacrylamides, polyvinyl ethers, hydroxypropyl cellulose, polyvinyl morpholinone, polymers and copolymers of vinyl sulfonic acid, polyacrylates, polyacrylamides, polyvinyl pyridine and the like. Other suitable polymers can include hydrolyzed acrylonitrile grafted starch, acrylic acid grafted starch, and isobutylene maleic anhydride copolymers, and mixtures thereof. Organic high-absorbency materials can include natural materials, such as agar, pectin, guar gum and peat moss. In addition to organic materials, the SAP materials may also include inorganic materials, such as absorbent clays and silica gels. The SAP material can comprise fibers and/or particles that may be spherical, spherical-like or irregularly shaped particles, such as sausage shaped particles, or ellipsoid shaped particles of the kind typically obtained from inverse phase suspension polymerizations. The SAP particles can also be optionally agglomerated at least to some extent to form larger irregular particles. In some embodiments, the SAP particles can also have a surface modification, such as a partial or full surface coating, for example to increase the hydrophilicity of the SAP particles.

Any embodiment of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Further, a device or system that is configured in a certain way is configured in at least that way, but it can also be configured in other ways than those specifically described.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1A is a bottom plan view of a prior art disposable absorbent article, specifically adult protective underwear, in an open configuration.

FIG. 1B is a perspective view of the protective underwear of FIG. 1A in a closed configuration.

FIGS. 2A and 2B are a top plan view and an exploded perspective view, respectively, of a first embodiment of an absorbent article, which is an underpad.

FIG. 2C is a sectional view of the underpad of FIG. 2A taken along line 2C-2C and illustrates the structure of the underpad's absorbent laminates.

FIG. 3A is a top plan view of a second embodiment of an absorbent article that is an underpad and has two differently-sized laminates.

FIG. 3B is a sectional view of the underpad of FIG. 3A taken along line 3B-3B and illustrates the manner in which one of the laminates of the underpad is folded. For clarity,

FIG. 3B does not depict the individual laminae of the laminates shown therein.

FIGS. 4A-4C are sectional views of third, fourth, and fifth embodiments of an absorbent article, each having different folded laminate configurations. For clarity, FIGS. 4A-4C do not depict the individual laminae of the laminates shown therein.

FIG. 5 is a bottom plan view of a sixth embodiment of an absorbent article, specifically adult protective underwear, that includes pads and is in an open configuration.

FIG. 6A is a top plan view of a seventh embodiment of an absorbent article, specifically an underpad, in which the topsheet comprises first and second topsheet layers and at least a portion of the first topsheet layer is configured to slide relative to the second topsheet layer within a slidable region of the topsheet.

FIGS. 6B and 6C are sectional views of the article of FIG. 6A taken along line 6B-6B and illustrate the gathered construction of the first topsheet layer when it is in a relaxed state (FIG. 6B) and an extended state (FIG. 6C).

FIGS. 7A and 7B are graphs showing conductivity measurements of samples of some of the present underpads and two commercial underpads after the samples were wetted with a saline solution. The conductivity measurements are representative of surface moisture.

FIGS. 8A-8D and 9A-9B are charts showing the results of interface pressure tests for some of the present underpads and one commercial underpad.

FIGS. 10A and 10B are charts showing lateral air permeability and z-direction air permeability, respectively, of samples of some of the present underpads and two commercial underpads.

FIGS. 11A and 11B are charts showing cross direction and machine direction lateral displacements, respectively, of fingers moving apart on surface of a sample immediately before the fingers slip on the surface. The displacements shown are for samples of some of the present underpads and three commercial underpads

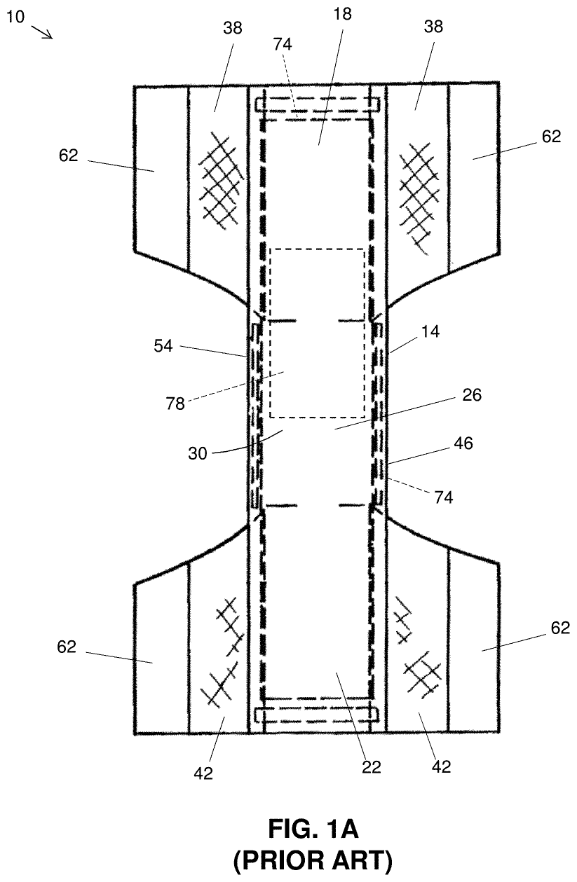

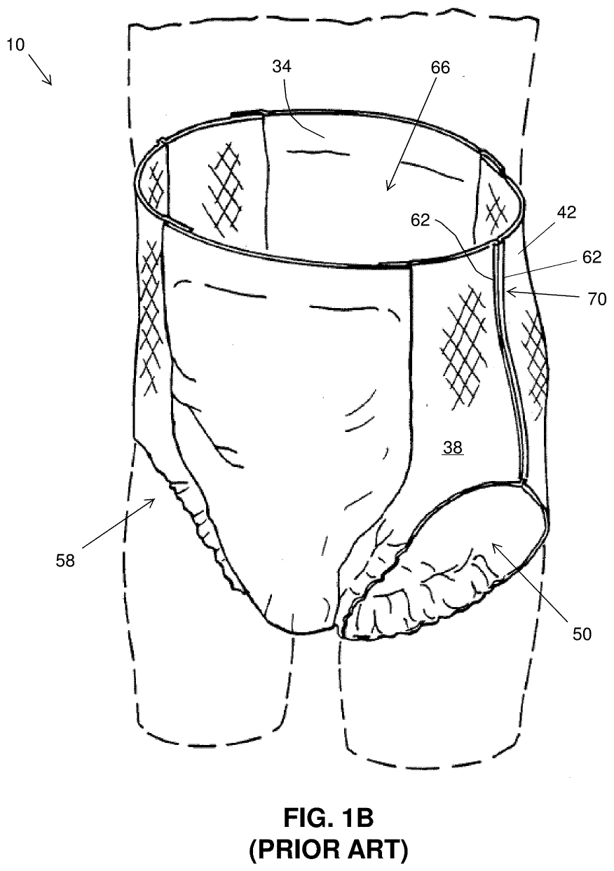

### DETAILED DESCRIPTION OF ILLUSTRATIVE EMBODIMENTS

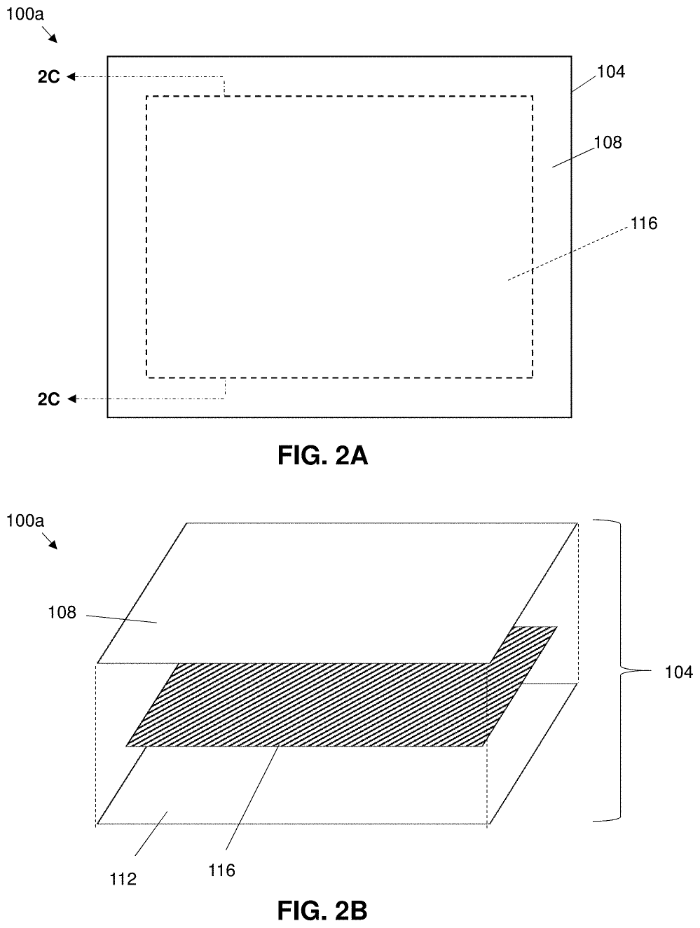

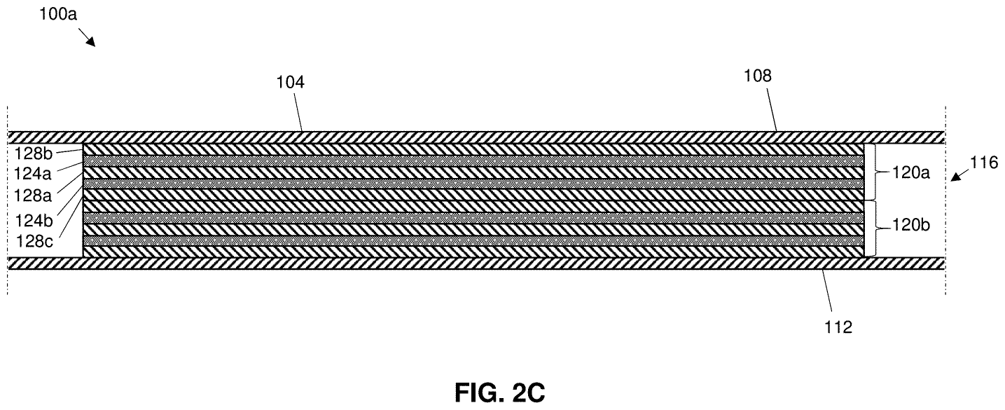

Referring to FIGS. 2A-2C, shown is a first embodiment 100a of the present absorbent articles. Article 100a, as shown, is an underpad; however, in other embodiments, the article can be a bed pad, seat cushion, patient support pad, breast pad, bandage, baby diaper, training pant, adult incontinence brief or underwear, bladder control pad, feminine hygiene pad, or the like. Article 100a can comprise a chassis 104 having a liquid-permeable topsheet 108 configured to face a user and a liquid-impermeable backsheet 112 configured to face away from the user during use of article 100a. For example, backsheet 112 can include an inner liquid-impermeable film and an outer nonwoven cover, and can be breathable, e.g., the liquid-impermeable film can comprise a breathable film.

In some embodiments, chassis 104 can omit backsheet 112, the backsheet can be liquid-permeable, and/or the backsheet can comprise a nonwoven without a liquid-impermeable film. Liquid-impermeable films, even if breathable (e.g., with respect to vapor), may have little, if any, air permeability. Backsheet 112, when comprising a nonwoven without a liquid-impermeable film, can be air-permeable to facilitate airflow through article 100a and thereby promote skin health and reduce the risk of pressure ulcers, as discussed in further detail below. Such a nonwoven backsheet 112 can comprise multiple layers, where at least two of the layers have different nonwoven constructions. For example, nonwoven backsheet 112 can comprise a meltblown nonwoven layer disposed between first and second spunbond nonwoven layers, where, optionally, the meltblown nonwoven layer constitutes greater than or equal to any one of, or between any two of, 10%, 15%, 20%, 25%, 30%, 35% or more (e.g., between 10% and 25%) of the nonwoven backsheet. Such a construction may provide suitable air permeability while impeding leakage through backsheet 112 (e.g., the backsheet can be liquid-impermeable).

Article 100a can include an absorbent core 116 disposed between topsheet 108 and backsheet 112. Topsheet 108 and core 116 can interact to promote dryness at the user-facing surface of the topsheet, improve breathability, and facilitate reductions in TIP. The respective constructions of topsheet 108 and core 116, including the materials used and the arrangement of the component(s) thereof, can affect the extent to which these benefits are achieved. Appropriate selection of the materials and the arrangement of components for topsheet 108 and core 116 can produce synergistic effects in which dryness, breathability, and TIP reductions are promoted to a greater extent than would be expected based on the respective topsheet and core constructions alone.

To achieve such synergistic benefits, topsheet 108 preferably comprises a high-bulk nonwoven, e.g., a nonwoven having a bulk greater than or equal to any one of, or between any two of, 8.5 cm³/g, 10 cm³/g, 12 cm³/g, 14 cm³/g, 16 cm³/g, 18 cm³/g, 20 cm³/g, 22 cm³/g, or higher (e.g., greater than or equal to 10 cm³/g) and core 116 preferably comprises one or more absorbent laminates (e.g., 120a, 120b). As used herein, bulk refers to the inverse of the bulk density of the nonwoven; bulk density can be calculated by dividing the basis weight of the nonwoven by the thickness thereof. The nonwoven of topsheet 108 can have a basis weight that is greater than or equal to any one of or between any two of 10 grams per square meter (gsm), 20 gsm, 30 gsm, 40 gsm, 50 gsm, 60 gsm, 70 gsm, 80 gsm, 90 gsm, 100 gsm, 110 gsm, 120 gsm, or more (e.g., between 10 and 120 gsm, such as between 50 and 120 gsm) and a thickness that is greater than or equal to any one of, or between any two of 0.40 mm, 0.60 mm, 0.80 mm, 0.90 mm, 1.10 mm, 1.30 mm, 1.50 mm, 1.70 mm, or larger (e.g., greater than or equal to 0.50 mm). Reductions in TIP may be relatively greater when topsheet 108 has a comparatively high basis weight (e.g., greater than or equal to 50 gsm) and/or thickness (e.g., greater than or equal to 0.50 mm), compared to lighter and thinner topsheets. The thickness and basis weight of a nonwoven can be measured pursuant to Nonwovens Standard Procedures—Edition 2015 (European Disposables and Nonwovens Association (EDANA) and the Association of the Nonwovens Fabrics Industry (INDA)) method numbers NWSP 120.1.R0 (15) and NWSP 130.1.R0 (15), respectively, which are hereby incorporated by reference. As described in further detail below, the suitability of a basis weight for high-bulk nonwoven topsheet 108 can depend at least in part on the construction of the laminate(s) of core 116. Suitable nonwoven materials can include, for example, spunbond, spunlace, or carded webs of one or more polymers, including polypropylene (PP), polyethylene (PE), polyethylene terephthalate (PET), nylon, polyester, and blends of these materials. Additionally or alternatively, such nonwoven materials can comprise wettable, regenerated cellulosic fibers such as, for example, viscose or Tencel®, either alone or in a blend with one or more of the above-described polymers. Nonwoven topsheet 108 can be embossed, comprise apertures, and/or be treated (e.g., with a surfactant).

Each of the laminate(s) of core 116 can comprise one or more absorbent laminae (e.g., 124a and 124b) and one or more substrate laminae (e.g., 128a-128c), each of which can have substantially the same thickness or different thicknesses. Each of the absorbent lamina(e) can comprise SAP particles to absorb liquid insults. The basis weight of the SAP particles in each of the absorbent lamina(e) can be greater than or equal to or between any two of 20, 30, 40, 50, 60, 70, 80, 90, 100, 110, 120, 130 or more gsm (e.g., between 20 and 130 gsm). The suitability of a SAP basis weight can depend at least in part on the number of laminates, the number of absorbent laminae in each of the laminates, and the desired absorption capacity of core 116. To illustrate, a suitable total basis weight of SAP particles in core 116 can be greater than or equal to 160 gsm (e.g., greater than or equal to 200 gsm or 300 gsm). As shown, core 116 comprises two laminates 120a and 120b, each comprising two absorbent laminae 124a and 124b such that the core has four absorbent laminae. As such, to achieve the desired SAP content in core 116, for each of laminates 120a and 120b, the SAP particles of each of absorbent laminae 124a and 124b can have a basis weight greater than or equal to, or between any two of, 20 gsm, 30 gsm, 40 gsm, 50 gsm, 60 gsm, 70 gsm, 80 gsm, or more (e.g., between 40 and 60 gsm or between 65 and 85 gsm). SAP particles having a comparatively lower basis weight (e.g., between 40 and 85 gsm) can maintain the resiliency of core 116 to reduce the TIP between article 100a and a user.

Exemplary superabsorbent polymer material suitable for use in the present articles can comprise any superabsorbent polymer particles known from superabsorbent literature, for example such as described in Modern Superabsorbent Polymer Technology, F. L. Buchholz, A. T. Graham, Wiley 1998. Suitable examples of SAP include T9030, T9600, T9900, and Saviva polymers from BASF Corporation in Charlotte, N.C.; and W211, W112A, W125, S125D, QX-W1482, QX-W1486, QX-W1504, and QX-W1505 from Nippon Shokubai Co. Ltd, N.A.I.I. in Houston, Tex.; and AQUA KEEP SA50 II, SA55SX II, SA60N II, SA65s, HP500, HP500E, HP600, and HP 700E from Sumitomo Seika Chemicals Co., Ltd. in Osaka, Japan. The SAP particles of each of the absorbent lamina(e) can have a particle size distribution (PSD) with most or substantially all of the particles having a diameter between 45 micrometers (μm) and 4000 μm, optionally between 150 μm and 850 μm. To promote comfort, substantially all of the SAP particles in at least one (e.g., each) of the absorbent lamina(e) can have a diameter less than or equal to 500 μm to reduce the roughness of the laminate. For example, ones of the SAP particles in an absorbent lamina having a diameter greater than or equal to 500 μm can account for less than 10% (e.g., less than 2% or less than 0.2%) of the mass of the SAP particles in the lamina. As used herein, particle diameter refers to the equivalent diameter of the particle if the particle is modelled as a sphere, and the PSD of a particulate material can be determine, for example, by means of dry sieve analysis (EDANA 420.02 Particle Size distribution). The SAP particles of each of the absorbent lamina(e) can have a comparatively high sorption capacity. To illustrate, the SAP particles can have a centrifuge retention capacity that is greater than or equal to or between any two of 20, 30, 40, 50, 60 or higher grams per gram (g/g) (e.g., between 20 and 60 g/g or between 40 and 50 g/g).

For each of the absorbent lamina(e), the SAP particles can be disposed within a matrix of adhesive material. Suitable adhesive material can include a thermoplastic hot-melt adhesive composition or a pressure-sensitive thermoplastic adhesive composition. Each of the absorbent lamina(e) can comprise at least 90% (e.g., greater than 93% or 94%), by weight, SAP and less than or equal to 10% (e.g., less than 6% or 7%), by weight, adhesive. For example, the basis weight of the adhesive in each of the absorbent lamina(e) can be greater than or equal to or between any two of 0.5 gsm, 1 gsm, 2 gsm, 3 gsm, 4 gsm, 5 gsm, 6 gsm, 7 gsm, 8 gsm, 9 gsm, 10 gsm, or more (e.g., between 0.5 and 10 gsm). The adhesive can be present in sufficient quantities such that the elongation at break of the laminate is at least 100% (e.g., between 600% and 1800%) to reduce gel blocking when the SAP particles are swollen. The adhesive can be porous to facilitate fluid transfer throughout the absorbent lamina and to promote the compressibility and resilience of the laminate, thereby facilitating a reduction in TIP.

When wetted, the SAP particles can act similarly to a bladder, retaining fluid under pressure and resiliently deforming and interacting with adjacent SAP particles to distribute forces between article 100a and a user over a larger area, thereby reducing TIP. Article 100a can be configured to permit pre-hydration of the SAP particles (e.g., with water or saline, before bodily exudates are absorbed) to improve softness and resiliency. For example, article 100a can optionally comprise a rupturable bladder disposed between topsheet 108 and backsheet 112 that can hold a volume of liquid sufficient to, when absorbed by the SAP particles of laminates 120a and 120b, swell at least a portion of the SAP particles to a desired resiliency. Additionally or alternatively, article 100a can optionally comprise an inlet (e.g., a sealable inlet) through which liquid can be introduced between topsheet 108 and backsheet 112 into core 116.

When one of the laminate(s) has multiple absorbent laminae, the absorbent laminae can be substantially the same or different. For example, at least two of the absorbent laminae can have different SAP properties (e.g., SAP basis weight, PSD, and/or centrifuge retention capacity) and/or different adhesive properties (e.g., adhesive basis weight).

Each of the substrate lamina(e) can be constructed from a nonwoven material and/or tissue to facilitate liquid acquisition and distribution throughout core 116 and thereby mitigate liquid accumulation at the user-facing surface of topsheet 108. Suitable nonwoven materials for the substrate lamina(e) include, for example, any of the webs of polymers and/or regenerated cellulosic fibers described above with respect to topsheet 108, and can have a basis weight greater than or equal to or between any two of 2 gsm, 10 gsm, 20 gsm, 30 gsm, 40 gsm, 50 gsm, 60 gsm, or more (e.g., between 20 and 40 gsm or between 40 and 60 gsm). Suitable tissue materials for the substrate lamina(e) include, for example, through-air dried (TAD) and/or creped tissue. When constructed from a tissue, a substrate lamina can have a basis weight greater than or equal to, or between any two of, 15 gsm, 20 gsm, 25 gsm, 30 gsm, 35 gsm, or more.

To facilitate liquid acquisition and distribution throughout core 116, each of the laminate(s) can be constructed such that each of the substrate lamina(e) is adjacent to at least one of the absorbent lamina(e). For example, as shown, each of laminates 120a and 120b comprises two absorbent laminae 124a and 124b and three substrate laminae 128a-128c. First substrate lamina 128a can be disposed between and in contact with first and second absorbent laminae 124a and 124b, the first absorbent lamina can be disposed between and in contact with the first substrate lamina and second substrate lamina 128b, and the second absorbent lamina can be disposed between and in contact with the first substrate laminae and third substrate lamina 128c. When constructed from a nonwoven, a substrate lamina can absorb and distribute rapid insults of liquid to the adjacent absorbent lamina(e), thereby mitigating gel blocking and reducing liquid accumulation at the user-facing surface of topsheet 108 during rapid insults of liquid. Substrate lamina(e) constructed from a tissue can provide a capillary network through which liquid is spread to the adjacent absorbent lamina(e) to further mitigate gel blocking, maintain an adequate acquisition rate, and reduce liquid accumulation at the user-facing surface of topsheet 108.

To illustrate, first substrate lamina 128a can comprise a nonwoven (e.g., a spunlace nonwoven), where at least a portion (e.g., all) of the fibers of the nonwoven can comprise viscose fibers and the nonwoven can have a basis weight between 20 and 40 gsm, optionally between 25 and 35 gsm. Such a first substrate lamina 128a can effectively facilitate liquid transfer from first absorbent lamina 124a (e.g., the upper adjacent absorbent lamina) to second absorbent lamina 124b (e.g., the lower adjacent absorbent lamina). First substrate lamina 128a can accordingly mitigate the tendency of first absorbent lamina 124a to restrict downward flow to second absorbent lamina 124b. As such, first substrate lamina 128a can facilitate wicking to promote dryness at the user-facing surface of topsheet 108, even though the first substrate lamina is not adjacent to the topsheet.

At least one of (e.g., each of) second and third substrate laminae 128b and 128c can comprise a tissue (e.g., a TAD tissue) to promote liquid distribution throughout core 116. Additionally or alternatively, at least one of second and third substrate laminae 128b and 128c can comprise a nonwoven (e.g., a spunlace nonwoven). For example, one of second and third substrate laminae 128b and 128c can comprise a nonwoven (hereinafter, the “outer nonwoven”), and the other of the second and third substrate laminae can comprise a tissue. The outer nonwoven can be different than that used for first substrate lamina 128a. For example, the outer nonwoven can have a higher basis weight than that of first substrate lamina 128a, e.g., a basis weight that is between 40 and 60 gsm, optionally between 45 and 55 gsm. Not to be bound by any particular theory, the higher basis weight of the outer nonwoven can promote the structural integrity of the laminate when the laminate is wetted, while also facilitating liquid transport and promoting resiliency to reduce TIP. At least a portion of the fibers of the outer nonwoven can comprise viscose; for example, the outer nonwoven can comprise a blend of viscose fibers and polymer fibers (e.g., PET fibers). Such a blend can comprise any suitable proportion of fibers, such as between 40% and 60% viscose fibers and between 40% and 60% polymer fibers, by weight.

For at least one of the laminate(s) (e.g., for first laminate 120a, which is disposed closer to topsheet 108 than is second laminate 120b), second substrate lamina 128b can be disposed on the topsheet such that the second substrate lamina is positioned closer to the topsheet than is third substrate lamina 128c. The interaction between second substrate lamina 128b and topsheet 108, and the respective constructions thereof, can affect the ability of core 116 to transport liquid insults away from the user-facing surface of topsheet 108. As described above, second substrate lamina 128b can comprise a tissue or a nonwoven (e.g., the outer nonwoven); unexpectedly, while each of these materials can facilitate liquid transport, the performance of the materials can depend at least in part on the construction (e.g., the basis weight and/or material(s)) of topsheet 108.

For at least some embodiments, when topsheet 108 comprises a comparatively heavy high-bulk nonwoven (e.g., a nonwoven having a basis weight between 65 and 120 gsm, optionally between 70 and 120 gsm), first laminate 120a can better encourage more and/or faster liquid removal from the user-facing surface of topsheet 108 when second substrate lamina 128b comprises tissue. In such embodiments, the nonwoven of topsheet 108 can comprise, for example, a needlepunch or spunlace nonwoven (e.g., comprising PE and/or PET fibers, optionally without viscose fibers), and can be embossed. The higher capillarity of the tissue of second substrate lamina 128b can promote faster transmission of moisture from the comparatively heavier topsheet 108 to first laminate 120a. When topsheet 108 comprises a comparatively lighter high-bulk nonwoven (e.g., a nonwoven having a basis weight between 10 and 65 gsm, optionally between 25 and 65 gsm), first laminate 120a can better encourage more and/or faster liquid removal from the user-facing surface of topsheet 108 when second substrate lamina 128b comprises a nonwoven. In such embodiments, the nonwoven of topsheet 108 can comprise, for example, a spunlace nonwoven (e.g., comprising PET and/or viscose fibers, optionally a blend of 20% to 40% viscose fibers and 60% to 80% PET fibers), and can be embossed and/or apertured. Article 100a can omit an ADL because the ADL may be unnecessary to achieve the desired fluid transportability.

The interaction between high-bulk nonwoven topsheet 108 and the laminate(s) of core 116 can also promote breathability. Due at least in part to the construction of topsheet 108 and the laminate(s), air and/or water vapor can diffuse laterally in chassis 104 (e.g., in direction a parallel with, rather than perpendicular to, the outer surface of topsheet 108 and/or of backsheet 112) to promote skin health. For example, topsheet 108 can facilitate airflow into core 116, where the air can flow laterally through the laminate(s) and moisture can be laterally diffused away from the location at which the user exerts pressure on article 100a (e.g., when lying on the article). Article 100a, when used, may sometimes be disposed between the user's skin and a non-breathable support surface (e.g., a bed or chair), which can limit air and/or water vapor diffusion through backsheet 112. The lateral breathability facilitated by the interaction between topsheet 108 and the laminate(s) of core 116 can provide an alternative flow path for air and/or water vapor to promote improved skin health, which may also reduce the risk of decubitus ulcers. Backsheet 112, when air-permeable (e.g., when comprising a nonwoven without a liquid-impermeable film), can facilitate such air flow as well.

High-bulk nonwoven top sheet 108 and the laminate(s) of core 116, due at least in part to their resiliency, can also facilitate reductions in TIP to reduce a user's risk of developing decubitus ulcers. For example, when topsheet 108 comprises a comparatively heavy high-bulk nonwoven (e.g., having a basis weight between 50 and 120 gsm, optionally 70 and 120 gsm), the TIP between article 100a and a user, when the article is disposed on a dry support surface (e.g., a mattress), may be lower than that which might have otherwise resulted if the user was placed directly on the support surface. By contrast, placing a conventional absorbent article between the user and the support surface may increase TIP by 20-25%. Additionally, high-bulk nonwoven topsheet 108 can reduce shear between a user's skin and the topsheet, relative to conventionally-used nonwoven topsheets. User movement can cause a surface of high-bulk nonwoven topsheet 108 to laterally displace (e.g., relative to the other components of article 100a) such that little, if any, slippage occurs between the topsheet and the user's skin. The amount of such displacement that may occur before shearing results can be relatively larger for high-bulk nonwovens and, as such, high-bulk nonwoven topsheet 108 can accommodate comparatively larger user movements to reduce shearing. Such a reduction in shear can further reduce the user's risk of developing decubitus ulcers.

As shown, each of laminates 120a and 120b comprises two absorbent laminae 124a and 124b and three substrate laminae 128a-128c. In other embodiments, each of the laminate(s) of core 116 can have any suitable number of absorbent laminae and substrate laminae arranged in any suitable order, such as, for example, greater than or equal to or between any two of 1, 2, 3, 4, 5, 6, 7, 8, or more absorbent laminae (e.g., comprising SAP particles and/or adhesive) and greater than or equal to or between any two of 1, 2, 3, 4, 5, 6, 7, 8, or more substrate laminae (e.g., comprising a nonwoven or a tissue). The number of substrate and absorbent laminae can be selected such that core 116 comprises a desired amount of SAP particles, exhibits a suitable resiliency to reduce TIP, and has sufficient strength when wetted. Any two adjacent laminae in each of the laminate(s) can be the same type of laminae (e.g., both can be absorbent laminae or substrate laminae) or laminae of different types (e.g., one can be one of the substrate lamina(e) and one can be one of the absorbent lamina(e)). By way of illustration, at least one of laminates 120a and 120b can omit second and third substrate laminae 128b and 128c. And, while core 116 comprises two laminates 120a and 120b, in other embodiments the core can comprise any suitable number of laminates, such as, for example, greater than or equal to or between any two of 1, 2, 3, 4, 5, 6, 7, 8, or more laminates (e.g., greater than or equal to 2 or 3 laminates). Providing additional laminates can increase the resiliency of core 116 with little, if any, impact on the core's ability to transport moisture away from topsheet 108.

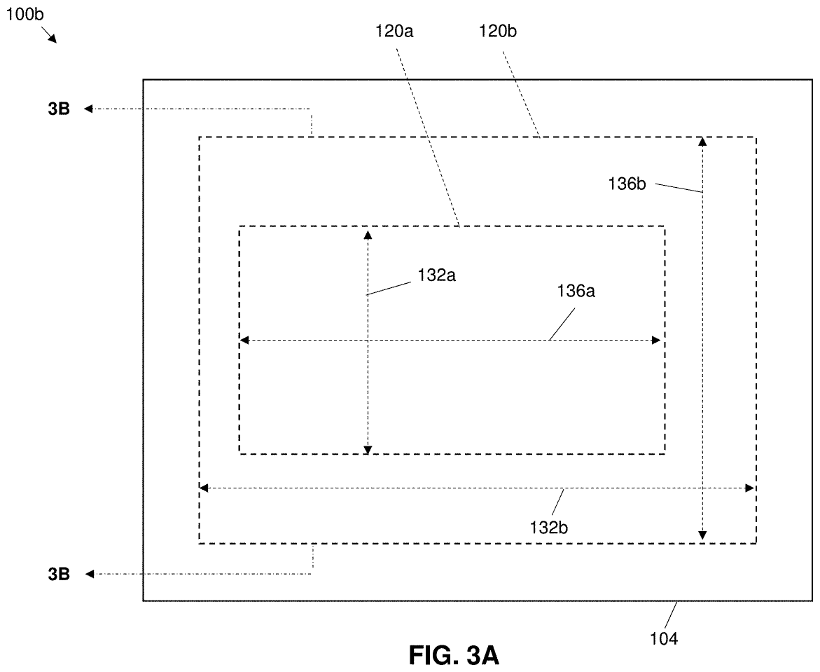

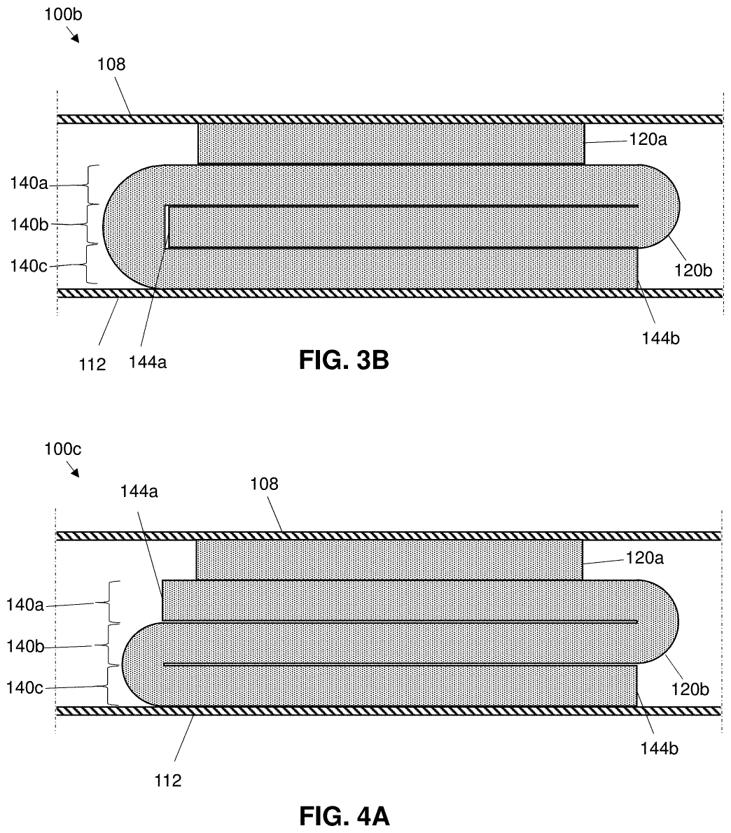

While laminates 120a and 120b of article 100a can be substantially the same size, in other multi-laminate embodiments the laminates can have different sizes. Referring to FIG. 3A, shown is an article 100b substantially similar to article 100a. Each of laminates 120a and 120b of article 100b can have a maximum width (e.g., 132a and 132b, respectively) measured in a first direction and a maximum length (e.g., 136a and 136b, respectively) measured in a second direction perpendicular to the first direction. Maximum width 132a of first laminate 120a can be smaller than (e.g., less than or equal to 90%, optionally between 50% and 70%, of) maximum width 132b of second laminate 120b, and/or maximum length 136a of the first laminate can be smaller than (e.g., less than or equal to 90%, optionally between 50% and 70%, of) maximum length 136b of the second laminate. When article 100b comprises an underpad or a bed pad, for example, maximum width 132b of second laminate 120b can be at least 12 inches and maximum length 136b of the second laminate can be at least 18 inches.

At least one of the laminate(s) of core 116 can be folded one or more times such that the laminate defines two or more layers. Referring to FIG. 3B, for example, second laminate 120b of article 100b can be folded such that the second laminate defines three layers 140a-140c, where second layer 140b is disposed between first and third layers 140a and 140c. Channels can be defined between adjacent ones of layers 140a-140c to facilitate lateral fluid flow between the layers and thereby encourage absorption by the absorbent lamina(e). The SAP particles of the absorbent lamina(e) can affect the surface topography of each of layers 140a-140c, e.g., by defining a plurality of valleys and micro-channels thereon that can encourage fluid dispersion between adjacent layers. SAP particles having comparatively larger particles sizes and higher gel stiffnesses can increase such surface topography. The folded construction of second laminate 120b can also promote the structural integrity thereof, including maintaining the relative positions of absorbent lamina(e) and substrate lamina(e), whether or not the absorbent lamina(e) and substrate lamina(e) are bonded to one another.

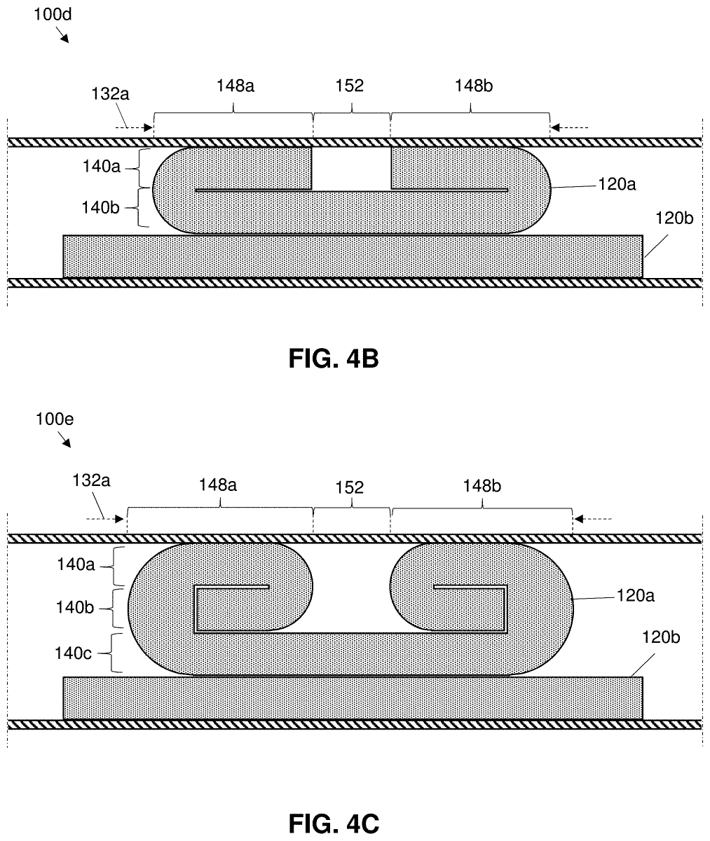

Second laminate 120b can be folded in any suitable manner. For example, second laminate 120b can be folded about one or more axes extending in a direction aligned with opposing widthwise edges 144a and 144b of the laminate. As shown, second laminate 120b is folded in a C-fold configuration in which second layer 140b comprises first edge 144a such that the first edge is disposed between first and third layers 140a and 140c. Referring to FIGS. 4A-4C, shown are articles 100c-100e, respectively, that are substantially similar to article 100b, the primary exception being the folded configuration of at least one of the laminate(s) of core 116. Second laminate 120b of article 100c, for example, can be folded in a Z-fold configuration in which third layer 140c comprises first edge 144a such that the edges of the second laminate are not disposed between layers thereof.

For each of articles 100d and 100e, first laminate 120a can be folded such that the first laminate defines one or more layers. For example, for article 100d, first laminate 120a can have a center region 152 disposed between first and second edge regions 148a and 148b, where a width of each of the edge regions is less than or equal to 50% of width 132a of the first laminate. First laminate 120a can be folded to define a base layer 140b and, within each of edge regions 148a and 148b, one or more layers (e.g., 140) disposed above the base layer such that a channel is defined within center region 152. Base layer 140b can span all of width 132a. First laminate 120a of article 100e can be folded in substantially the same manner as that of article 100d, except that as shown, within each of edge regions 148a and 148b, the first laminate defines two layers 140a and 140b disposed above base layer 140c, which can be, for example, a C-fold (e.g., as shown) or a Z-fold. Second laminate 120b, for each of articles 100d and 100e, can be not folded.

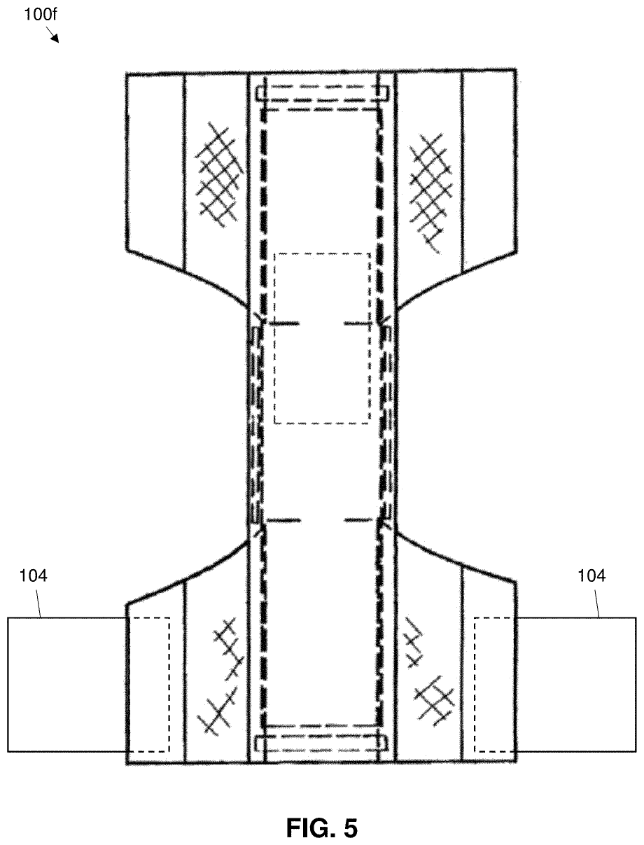

Referring to FIG. 5, shown is an article 100f that can be, for example, adult protective underwear, a diaper, or a training pant (e.g., substantially similar to underwear 10) which has pads, each comprising chassis 104. Each of the pads can have any of the constructions described above with respect to articles 100a-100e, and can be sized and shaped for use in article 100f. For example, the pads can be sized and shaped to extend around a wearer's hips, provide cushioning (e.g., to distribute forces exerted on the wearer), and reduce local maxima of TIP (e.g., to reduce the occurrence of bed sores). Additionally or alternatively, each of the pads can be sized and shaped to overlie a wearer's buttocks, and/or can define at least a portion of the chassis of article 100f (e.g., such that topsheet 108, backsheet 112, and core 116 define at least a portion of the topsheet, backsheet, and core of the underwear, respectively).

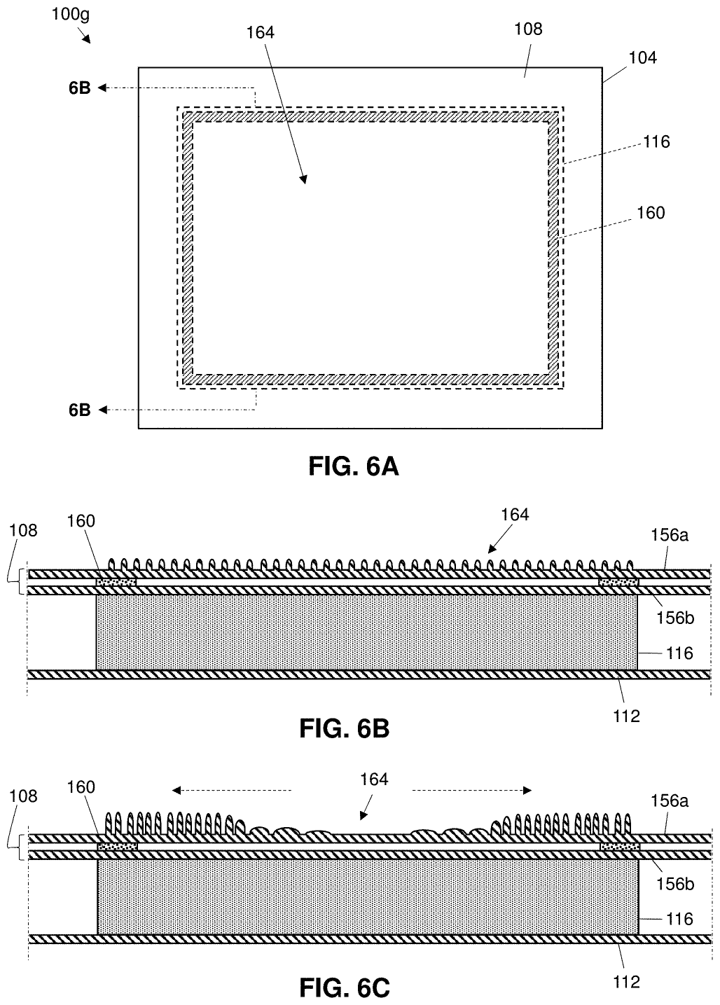

Referring to FIGS. 6A-6C, shown is an article 100g having a multi-layered topsheet 108. Article 100g can be a pad (e.g., an underpad, patient support pad, seat cushion, or the like) or any of the above-described absorbent articles, and can have an absorbent core 116—which can comprise one or more of the above-described laminate(s), fluff, and/or a fluff-SAP mixture—disposed between topsheet 108 and backsheet 112. Topsheet 108 can comprise first and second topsheet layers 156a and 156b, where the first topsheet layer is configured to face a user and is bonded to the second topsheet layer via adhesive 160. Topsheet layers 156a and 156b can be the same size or different sizes.

To reduce shear between a user's skin and topsheet 108, the topsheet can define a slidable region 164 that overlies at least a majority of core 116. Adhesive 160 can be disposed outside of slidable region 164 such that at least a portion of first topsheet layer 156a is slidable relative to second topsheet layer 156b within the slidable region. For example, adhesive 160 can comprise one or more portions, each of which extends along at least a portion of the perimeter of slidable region 164 (e.g., such that the adhesive surrounds at least a majority or all of the slidable region). Adhesive 160, as shown, comprises a continuous strip that surrounds all of slidable region 164, but in other embodiments the adhesive can comprise multiple separate portions.

First topsheet layer 156a can be configured to slide relative to second topsheet layer 156b in one or more directions, e.g., at least in a first direction aligned with a width of chassis 104 and in a second direction that is aligned with a length of the chassis and is perpendicular to the first direction. To illustrate, at least a portion of first topsheet layer 156a within slidable region 164 can be gathered (FIG. 6B) such that the first topsheet layer is extensible, and thus slidable, within the slidable region (FIG. 6C).

The coefficient of friction between first and second topsheet layers 156a and 156b can be lower than that between the first topsheet layer and the skin of a user such that movement of the user encourages sliding between the topsheet layers and thereby mitigates shear between the first topsheet layer and the user's skin. For example, each of first and second topsheet layers 156a and 156b can comprise a nonwoven. The reduced shearing between topsheet 108 and a user's skin can promote skin health and reduce the occurrence of bed sores.

### EXAMPLES

Absorbent articles of the present disclosure will be described in greater detail by way of specific examples. The following examples are offered for illustrative purposes only and are not intended to limit the invention in any manner. Those of skill in the art will readily recognize a variety of non-critical parameters that can be changed or modified to yield essentially the same results.

### Example 1

Eight prototypes of the underpad of FIGS. 2A-2C were produced and tested to assess rate of reduction in surface moisture of the prototypes. The backsheet of each of the prototypes comprised a 52 gsm spunbond nonwoven outer cover and a layer of BR-134 breathable poly film adhesively attached to the nonwoven. Both of the laminates of each of the prototypes had one of the two laminate constructions listed below in TABLE 1.

TABLE 1
Laminate Constructions of the Example 1 Prototypes

| | “Nonwoven Up” | “Tissue Up” |
|---|---|---|
| Second Substrate | 50 gsm, 50% viscose- | Tissue |
| Lamina | 50% PET spunlace | |
| | nonwoven | |
| First Absorbent | 75 gsm T9900 SAP | 75 gsm T9900 SAP |
| Lamina | | |
| First Substrate | 28 gsm, 100% viscose | 28 gsm, 100% viscose |
| Lamina | spunlace nonwoven | spunlace nonwoven |
| Second Absorbent | 75 gsm T9900 SAP | 75 gsm T9900 SAP |
| Lamina | | |
| Third Substrate | Tissue | 50 gsm, 50% viscose- |
| Lamina | | 50% PET spunlace |
| | | nonwoven |

The substrate lamina disposed on the topsheet was a nonwoven for the prototypes having the “Nonwoven Up” laminate configuration and a tissue for the prototypes having the “Tissue Up” laminate configuration. The prototypes also had different topsheets sourced from New England Nonwoven LLC (“NEN”), Spuntech Industries Inc. (“Spuntech”), Jacob Holm (“JH”), and Shalag U.S., Inc. (“Shalag”); TABLE 2 sets forth the topsheet materials and laminate configurations of the eight prototypes.

TABLE 2
Topsheet and Laminates Used in the Example 1 Prototypes

| | Topsheet | | | | |
|---|---|---|---|---|---|
| | | Measured | | | Laminate |
| | Description | Basis Weight | Thickness | Bulk | Configuration |
| Prototype 1 | NEN 3.0 osy PE needlepunch | 111 gsm | 1.47 mm | 13.2 cm³/g | Nonwoven Up |
| | nonwoven | | | | |
| Prototype 2 | NEN 3.0 osy PE needlepunch | 75.1 gsm | 1.19 mm | 15.8 cm³/g | Tissue Up |
| | nonwoven | | | | |
| Prototype 3 | Spuntech 60 gsm, 35% viscose- | 63.9 gsm | 0.551 mm | 8.6 cm³/g | Nonwoven Up |
| | 65% PET embossed spunlace | | | | |
| | nonwoven, waffle embossing | | | | |
| | pattern | | | | |
| Prototype 4 | JH 50 gsm, 35% viscose-65% | — | — | — | Nonwoven Up |
| | PET embossed spunlace | | | | |
| | nonwoven, dot embossing pattern | | | | |
| Prototype 5 | Shalag 50 gsm two-layered | — | — | — | Nonwoven Up |
| | nonwoven, through-air bonded | | | | |
| | ADL disposed therebetween | | | | |
| Prototype 6 | JH 30 gsm TRP aperture spunlace | — | — | — | Nonwoven Up |
| | nonwoven | | | | |
| Prototype 7 | Spuntech 75 gsm, 100% PET | — | — | — | Tissue Up |
| | embossed spunlace nonwoven, | | | | |
| | hills embossing pattern | | | | |
| Prototype 8 | Spuntech 60 gsm, 35% viscose- | 63.9 gsm | 0.551 mm | 8.6 cm³/g | Tissue Up |
| | 65% PET embossed spunlace | | | | |
| | nonwoven, waffle embossing | | | | |
| | pattern | | | | |

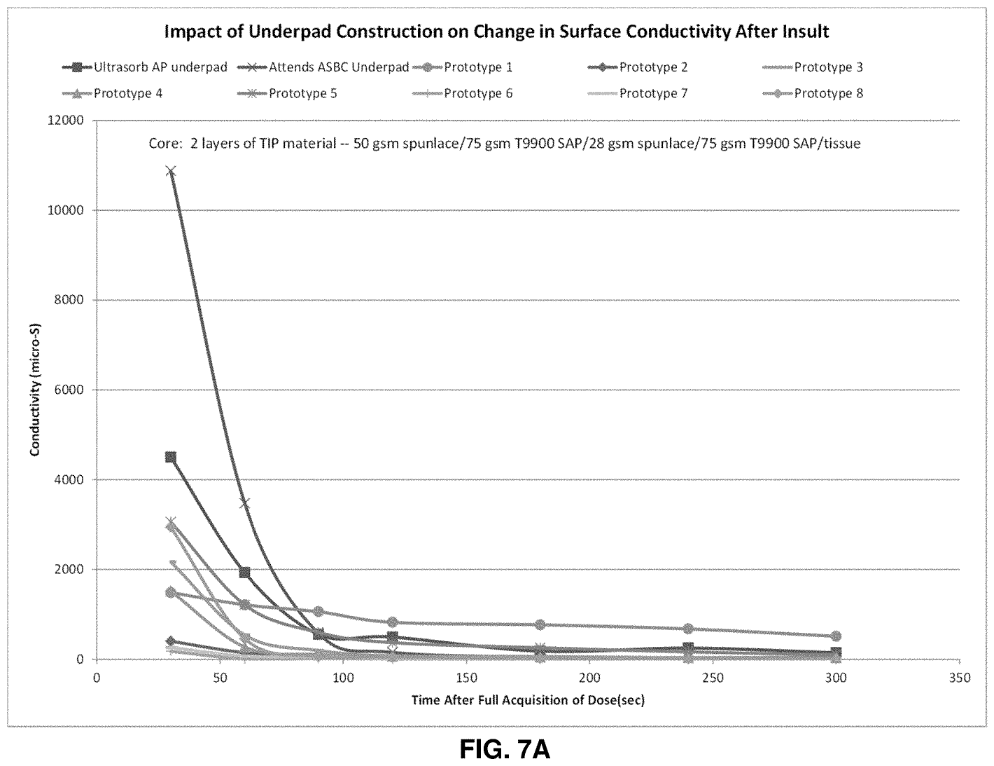

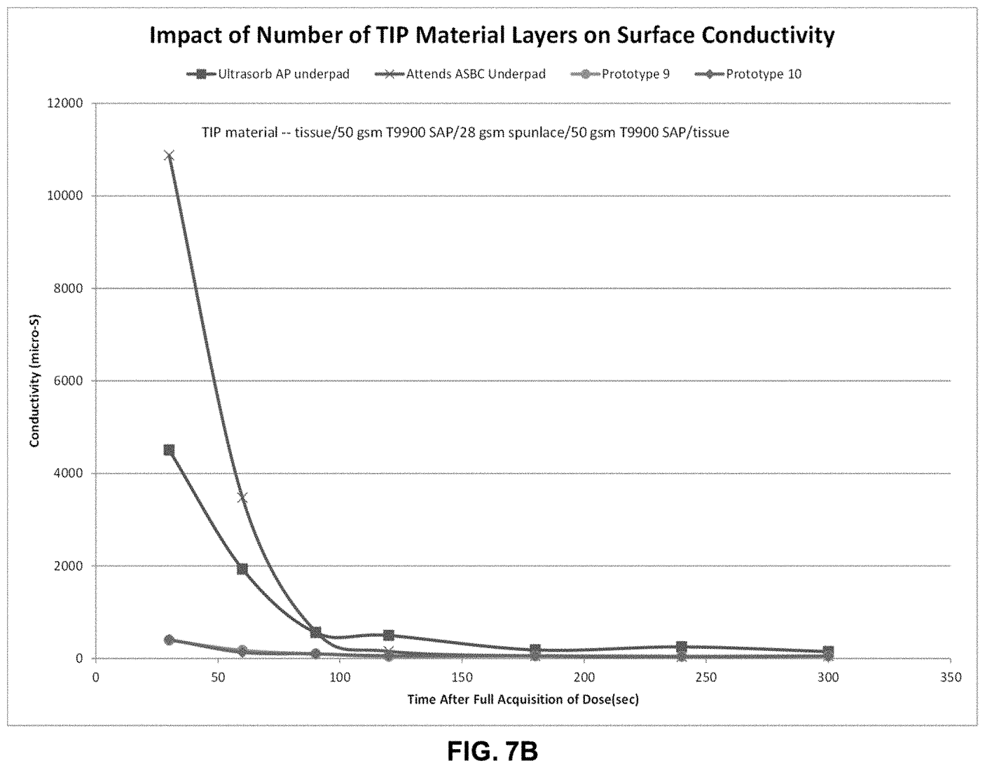

To perform the test, samples were cut from each of the prototypes—a 230 mm×190 mm sample was cut for each of Prototypes 1-2 and 4-7 and a 130 mm×250 mm sample was cut for each of Prototypes 3 and 8. For each sample, a 50 milliliter dose of 0.9% saline solution was applied to the topsheet at 40° C. and conductivity measurements were taken at the surface of the topsheet periodically between 30 seconds and 5 minutes after complete acquisition of the dose. Each of the periodic measurements included four measurements that were taken at different locations on the topsheet and averaged. A Cortex Technology DermaLab Hydration Pin Probe was used to take the conductivity measurements. The same test was performed for two commercial underpads: an ATTENDS® ALL-IN-ONE™ Complete Underpad (“ASBC”) and a MEDLINE ULTRASORBS® AP underpad (“Medline”), each of which was cut to produce a 250 mm×190 mm sample for testing. The results are shown in FIG. 7A, where lower conductivity measurements indicate lower amounts of surface moisture.

Each of the prototypes and commercial underpads exhibited a reduction in surface conductivity, and thus surface moisture, over the first two to three minutes following complete dose acquisition, with few changes after that time. All prototypes except for Prototype 1 and Prototype 5 exhibited lower surface moisture than the commercial underpads until about two minutes after complete dose acquisition, at which point the ASBC underpad achieved a similar reduction in surface moisture as the prototypes. Prototypes 2-4 and 6-8 accordingly demonstrated faster reductions in surface moisture than the commercial underpads.

Prototype 2 achieved faster reductions in surface moisture than Prototype 1 even though the only difference between the prototypes was that Prototype 1 had the Nonwoven Up laminate configuration and Prototype 2 had the Tissue Up laminate configuration. Additionally, until about one minute after full dose acquisition, Prototype 3 exhibited faster reductions in surface moisture than Prototype 8 even though the only difference between the prototypes was that Prototype 3 had the Nonwoven Up laminate configuration and Prototype 8 had the Tissue Up laminate configuration. Prototype 5 exhibited reduced surface moisture relative to the commercial underpads between 30 and 60 seconds after full dose acquisition, but had similar surface moisture content as the Medline underpad after 90 seconds. The tests demonstrated that the performance of the prototypes depended at least in part on the relationship between the topsheet and the laminate, rather than the construction of the topsheet or laminate alone. Of the prototypes tested, Prototype 2, Prototype 6, and Prototype 7 achieved the fastest reductions in surface moisture.

### Example 2

The test described in Example 1 was performed on two additional prototypes-Prototype 9 and Prototype 10—to assess the impact that increasing the number of laminates had on the rate of surface moisture reduction. Each of the prototypes was substantially the same as Prototype 2 of Example 1. The primary difference was that, for each of the laminates of each of Prototypes 9 and 10, both of the second and third substrate laminae comprised a tissue and each of the absorbent laminae comprised 50 gsm, rather than 75 gsm, SAP particles. Prototype 9 had two laminates (as in FIG. 2C) and Prototype 10 had three laminates. The results of the test are shown in FIG. 7B along with the conductivity data that was obtained for the commercial underpads in Example 1. There were no significant differences in surface moisture reductions between Prototype 9 and Prototype 10, both of which exhibited lower surface moisture than the commercial underpads until about 2 minutes after full dose acquisition.

### Example 3

Four prototypes were produced and tested to assess the impact of topsheet and laminate construction on TIP. Each of the prototypes had a topsheet disposed on three laminates, where each of the laminates had two absorbent laminae and three substrate laminae arranged as shown for the laminates in FIG. 2C. Two different laminate constructions were used in the prototypes, as set forth in TABLE 3.

TABLE 3
Laminate Constructions in the Example 3 Prototypes

| | “Tissue/Tissue” | “Nonwoven/Tissue” |
|---|---|---|
| Second Substrate | Tissue | 50 gsm, 50% viscose- |
| Lamina | | 50% PET spunlace |
| | | nonwoven |
| First Absorbent | 75 gsm T9900 SAP | 75 gsm T9900 SAP |
| Lamina | | |
| First Substrate | 28 gsm, 100% viscose | 28 gsm, 100% viscose |
| Lamina | spunlace nonwoven | spunlace nonwoven |
| Second Absorbent | 75 gsm T9900 SAP | 75 gsm T9900 SAP |
| Lamina | | |
| Third Substrate | Tissue | Tissue |
| Lamina | | |

TABLE 4 sets forth the topsheet materials and laminate arrangements of the tested prototypes.

TABLE 4
Topsheet Materials and Laminate Arrangements in the Example 3 Prototypes

| | | Upper | Middle | Lower |
|---|---|---|---|---|
| | Topsheet | Laminate | Laminate | Laminate |
| Prototype 11 | 12 gsm nonwoven | Nonwoven/ | Tissue/ | Tissue/ |
| | | Tissue | Tissue | Tissue |
| Prototype 12 | 12 gsm nonwoven | Nonwoven/ | Nonwoven/ | Nonwoven/ |
| | | Tissue | Tissue | Tissue |
| Prototype 13 | Spuntech 75 gsm, 100% PET | Nonwoven/ | Tissue/ | Tissue/ |
| | embossed spunlace nonwoven, | Tissue | Tissue | Tissue |
| | hills embossing pattern | | | |
| Prototype 14 | Spuntech 75 gsm, 100% PET | Nonwoven/ | Nonwoven/ | Nonwoven/ |
| | embossed spunlace nonwoven, | Tissue | Tissue | Tissue |
| | hills embossing pattern | | | |
| Prototype 15 | NEN 3.0 osy PE needlepunch | Nonwoven/ | Tissue/ | Tissue/ |
| | nonwoven | Tissue | Tissue | Tissue |

For each of the prototypes, testing was performed when the prototype was in a dry state, when the prototype was in a wet state, and on different surfaces: a hard surface, a foam pad, and a Span America UltraMax Geo-Mattress. Such testing simulated conditions that a user may experience during use. To begin, for each of the prototypes, the dry prototype was placed on the foam pad, a pressure mat was placed on the prototype, and a glass frit was placed on the pressure mat over the prototype with its convex side oriented toward the pressure mat and its flat side oriented towards the ceiling. A one-kilogram mass was placed on top of the glass frit. The area, average pressure, and maximum pressure at the interface between the frit and the topsheet were measured. The procedure was repeated for the hard surface and the mattress. After completing the test for the dry prototype on all surfaces, the entire procedure was repeated after wetting the prototype. To wet the prototype, 0.006 ml of tap water per square millimeter of the absorbent core was poured onto the topsheet of each of the prototypes. The test was also performed for the Medline underpad. The results are set forth in TABLE 5.

TABLE 5
Results from Example 3 TIP Testing

| | | | Surface | | |
|---|---|---|---|---|---|
| | Condition | | Hard | Foam | Mattress |
| Medline | Dry | Area (cm²) | 23.2 | 82.4 | 76.9 |
| Under- | | Average Pressure (mmHg) | 39.5 | 9.4 | 6.6 |
| pad | | Maximum Pressure (mmHg) | 147.3 | 28.8 | 17.7 |
| | Wet | Area (cm²) | 50.7 | 95.8 | 101.4 |
| | | Average Pressure (mmHg) | 11.5 | 6.3 | 5.0 |
| | | Maximum Pressure (mmHg) | 44.8 | 22.8 | 20.4 |
| Proto- | Dry | Area (cm²) | 23.2 | 61.2 | 65.5 |
| type 11 | | Average Pressure (mmHg) | 38.2 | 12.0 | 7.8 |
| | | Maximum Pressure (mmHg) | 123.7 | 30.9 | 24.7 |
| | Wet | Area (cm²) | 124.6 | 128.8 | 120.4 |
| | | Average Pressure (mmHg) | 4.1 | 3.1 | 3.3 |
| | | Maximum Pressure (mmHg) | 13.1 | 9.8 | 9.8 |
| Proto- | Dry | Area (cm²) | 29.6 | 63.3 | 78.1 |
| type 12 | | Average Pressure (mmHg) | 28.2 | 12.1 | 7.6 |
| | | Maximum Pressure (mmHg) | 82.4 | 32.4 | 26.7 |
| | Wet | Area (cm²) | 111.7 | 114.0 | 118.4 |
| | | Average Pressure (mmHg) | 3.4 | 3.5 | 3.6 |
| | | Maximum Pressure (mmHg) | 16.7 | 10.7 | 9.8 |
| Proto- | Dry | Area (cm²) | 52.8 | 76.0 | 90.8 |
| type 13 | | Average Pressure (mmHg) | 13.2 | 5.6 | 4.6 |
| | | Maximum Pressure (mmHg) | 61.7 | 19.4 | 11.7 |
| | Wet | Area (cm²) | 114.0 | 111.9 | 116.1 |
| | | Average Pressure (mmHg) | 3.8 | 3.7 | 4.2 |
| | | Maximum Pressure (mmHg) | 11.1 | 8.5 | 11.6 |
| Proto- | Dry | Area (cm²) | 52.8 | 82.4 | 97.1 |
| type 14 | | Average Pressure (mmHg) | 11.4 | 7.0 | 4.4 |
| | | Maximum Pressure (mmHg) | 30.7 | 21.6 | 11.3 |
| | Wet | Area (cm²) | 120.4 | 107.7 | 116.1 |
| | | Average Pressure (mmHg) | 4.8 | 4.0 | 3.7 |
| | | Maximum Pressure (mmHg) | 15.6 | 11.3 | 8.8 |
| Proto- | Dry | Area (cm²) | 57.0 | 73.9 | 105.6 |
| type 15 | | Average Pressure (mmHg) | 9.9 | 7.1 | 4.9 |
| | | Maximum Pressure (mmHg) | 27.6 | 25.9 | 26.1 |
| | Wet | Area (cm²) | 124.6 | 122.5 | 126.8 |
| | | Average Pressure (mmHg) | 3.5 | 2.6 | 2.4 |
| | | Maximum Pressure (mmHg) | 9.9 | 5.9 | 7.8 |

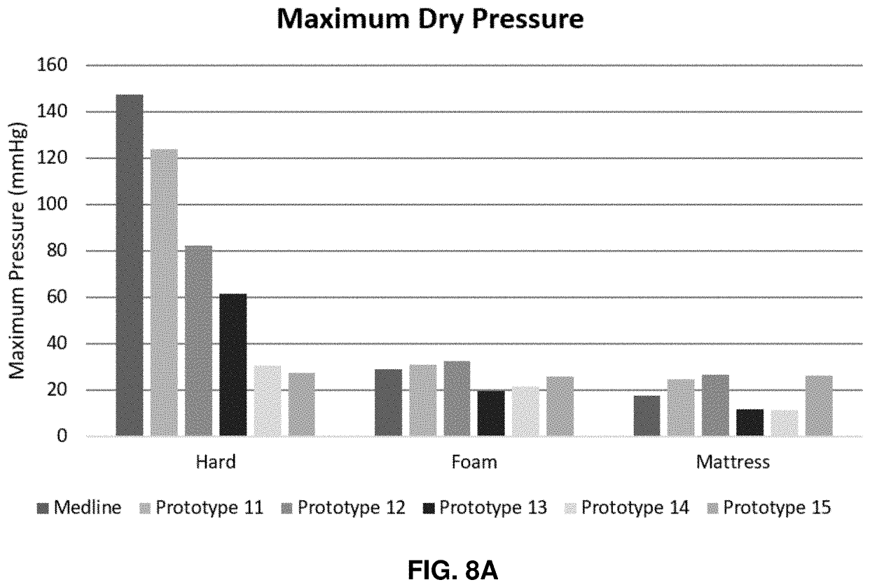

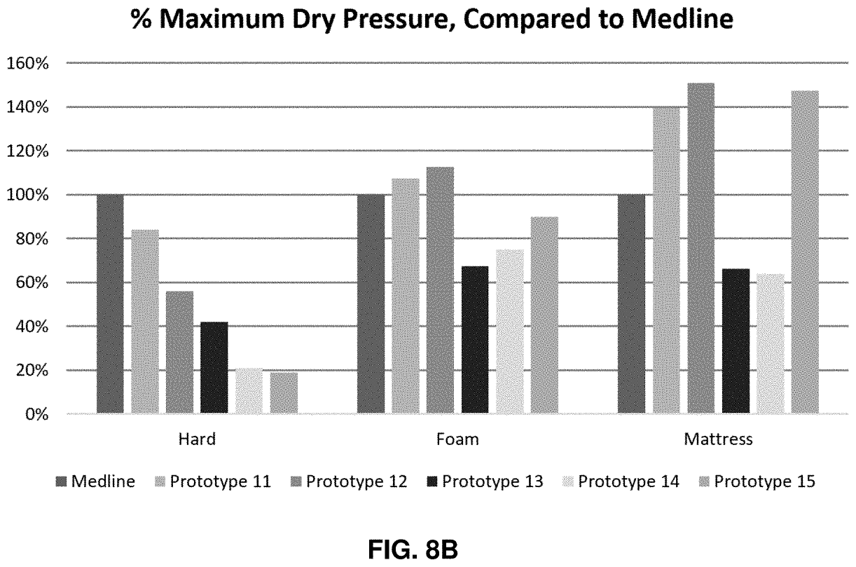

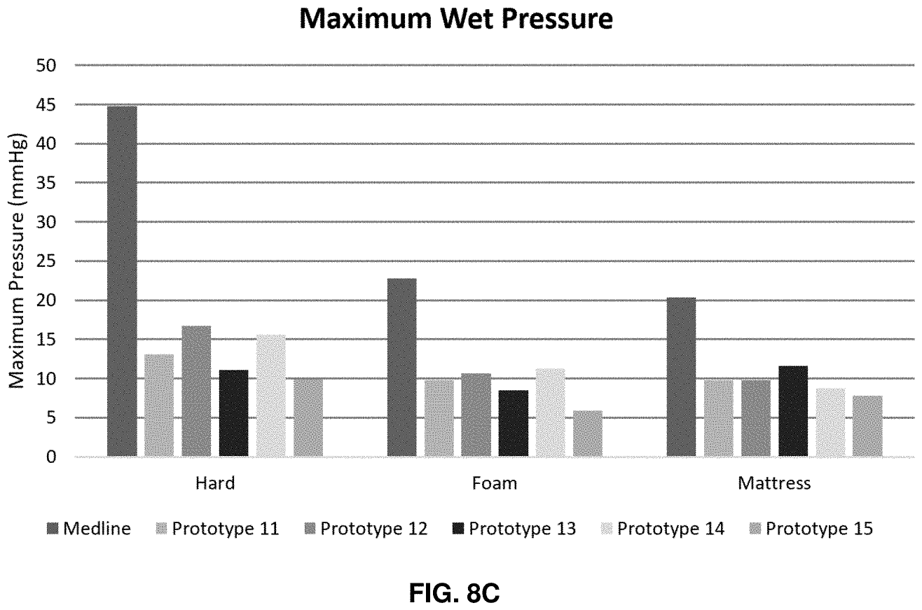

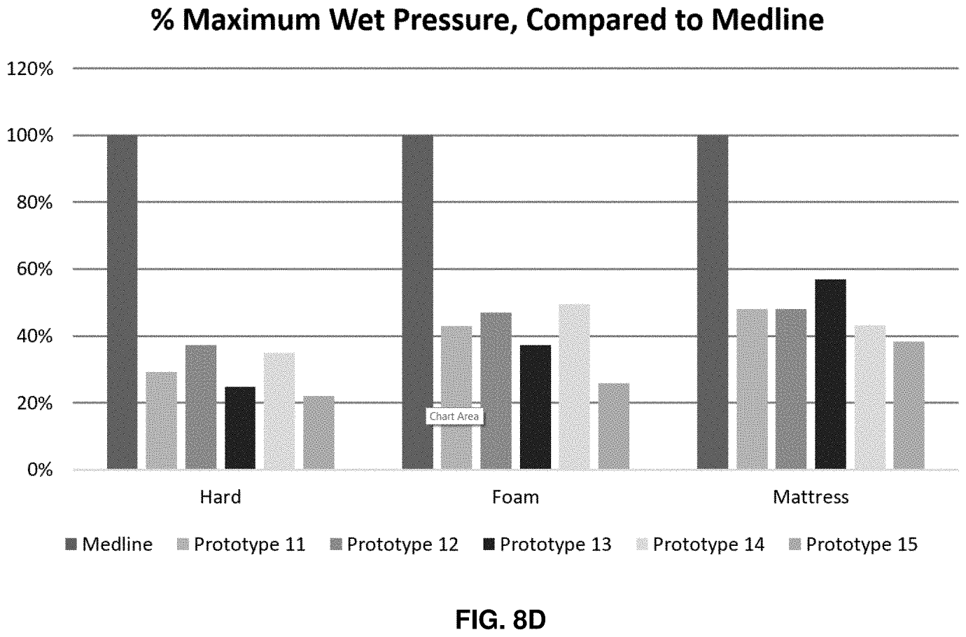

FIGS. 8A and 8C show the maximum pressures measured for each of the prototypes when the prototype was in the dry state and the wet state, respectively; FIGS. 8C and 8D normalize those results to the maximum pressure measured for the Medline underpad. When in a wet state, each of the prototypes exhibited reduced maximum interface pressure, compared to the Medline underpad, regardless of the type of surface. When in a dry state, each of the prototypes exhibited reduced maximum interface pressure relative to the Medline underpad when the prototype was disposed on the hard surface. However, only Prototype 13 and Prototype 14 achieved reduced pressure on all surfaces when in the dry state. Lower pressures were measured for Prototype 15, relative to the Medline underpad, when it was disposed on the foam pad but not when it was disposed on the mattress.

### Example 4

The test of Example 3 was repeated for the Medline underpad and Prototypes 11-13 and 15, except that a mannequin was used instead of the glass frit and weight to simulate a bony prominence. The results are set forth in TABLE 6.

TABLE 6
Results from Example 4 TIP Testing

| | | | Surface | | |
|---|---|---|---|---|---|
| | Condition | | Hard | Foam | Mattress |
| Medline | Dry | Area (cm²) | 147.8 | 435.1 | 612.4 |
| Under- | | Average Pressure (mmHg) | 117.6 | 43.2 | 28.3 |
| pad | | Maximum Pressure (mmHg) | 260.0 | 164.1 | 98.3 |
| | Wet | Area (cm²) | 219.6 | 420.9 | 656.7 |
| | | Average Pressure (mmHg) | 76.1 | 33.1 | 22.6 |
| | | Maximum Pressure (mmHg) | 260.0 | 109.5 | 75.5 |
| Proto- | Dry | Area (cm²) | 154.1 | 420.9 | 625.3 |
| type 11 | | Average Pressure (mmHg) | 120.3 | 39.2 | 26.3 |
| | | Maximum Pressure (mmHg) | 260.0 | 116.2 | 81.6 |
| | Wet | Area (cm²) | 251.4 | 500.3 | 682.0 |
| | | Average Pressure (mmHg) | 64.5 | 29.8 | 23.1 |
| | | Maximum Pressure (mmHg) | 260.0 | 97.6 | 77.5 |
| Proto- | Dry | Area (cm²) | 152.0 | 428.6 | 629.8 |
| type 12 | | Average Pressure (mmHg) | 120.9 | 41.7 | 25.6 |
| | | Maximum Pressure (mmHg) | 260.0 | 160.9 | 95.8 |
| | Wet | Area (cm²) | 221.7 | 443.5 | 618.7 |
| | | Average Pressure (mmHg) | 73.4 | 40.3 | 22.5 |
| | | Maximum Pressure (mmHg) | 260.0 | 130.9 | 71.0 |
| Proto- | Dry | Area (cm²) | 164.7 | 428.6 | 620.9 |
| type 13 | | Average Pressure (mmHg) | 104.5 | 39.1 | 24.0 |
| | | Maximum Pressure (mmHg) | 260.0 | 120.2 | 81.0 |
| | Wet | Area (cm²) | 257.6 | 439.7 | 629.2 |
| | | Average Pressure (mmHg) | 63.9 | 34.5 | 23.7 |
| | | Maximum Pressure (mmHg) | 260.0 | 107.8 | 77.9 |
| Proto- | Dry | Area (cm²) | 173.1 | 468.9 | 610.2 |
| type 15 | | Average Pressure (mmHg) | 92.6 | 37.6 | 25.0 |
| | | Maximum Pressure (mmHg) | 260.0 | 121.4 | 86.8 |
| | Wet | Area (cm²) | 270.3 | 464.7 | 684.2 |
| | | Average Pressure (mmHg) | 60.2 | 32.0 | 24.7 |
| | | Maximum Pressure (mmHg) | 260.0 | 93.3 | 76.8 |

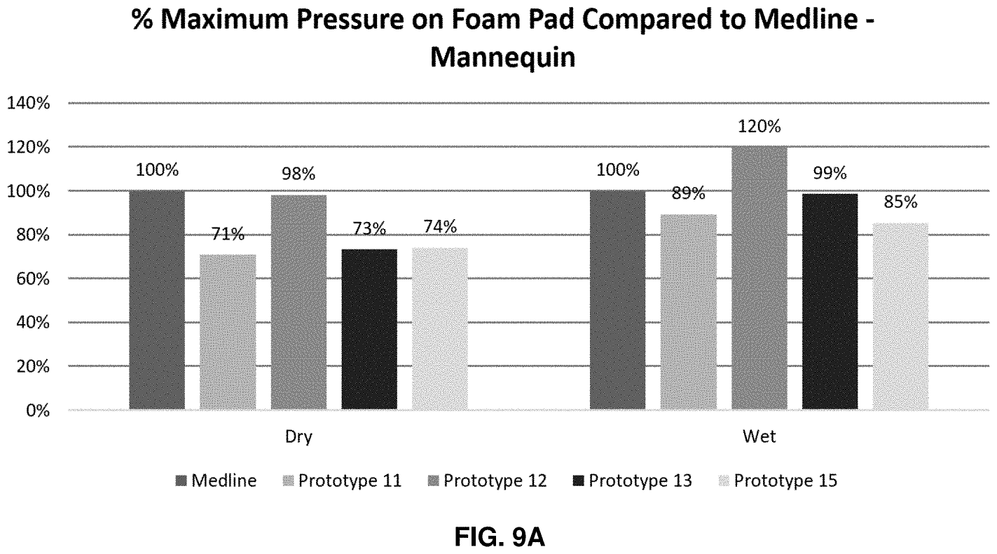

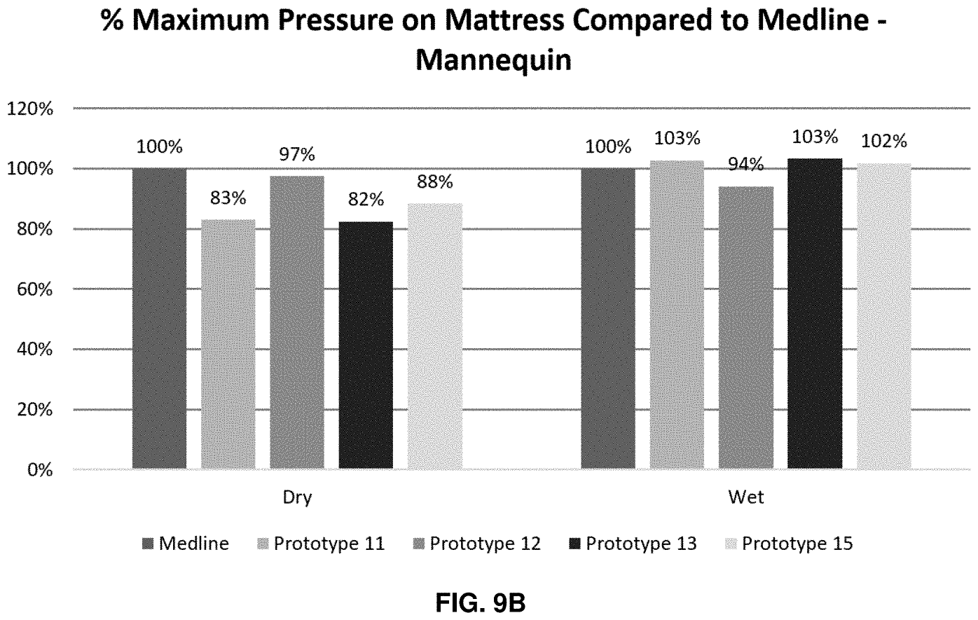

FIGS. 9A and 9B show the maximum pressures measured during the tests performed on the foam pad and mattress, respectively, normalized to the maximum pressure measured for the Medline underpad. When the tested prototypes were in the dry state, each of the tested prototypes exhibited reduced maximum interface pressures, compared to the Medline underpad, on both the foam pad and the mattress. When the tested prototypes were in the wet state, the maximum interface pressure measured for each of Prototype 11, Prototype 13, and Prototype 15 was lower than for the Medline underpad when disposed on the foam pad, and the maximum interface pressure measured for Prototype 12 was lower than for the Medline underpad when disposed on the mattress. The reductions in maximum interface pressure were not as significant as those measured in Example 3, potentially because the mannequin was not as rigid as the glass frit and thus distributed the loads more evenly across the underpads. For each of the tests performed on the hard surface the maximum pressure measured was 260 mmHg, which was the upper limit measurable by the pressure mat used in the tests.

### Example 5

The test of Example 3 was repeated for the Medline underpad, the ASBC underpad, and additional prototypes and samples set forth below in TABLE 7. The trials were limited to dry samples on the foam pad, dry samples on the mattress, and wet samples on the foam pad. The maximum interface pressure between the glass frit and mattress, e.g. without an underpad, was also measured. The maximum pressure measurements are set forth in TABLE 8. TABLE 8 also shows each of these maximum pressure measurements normalized to the maximum pressures measured for the Medline and ASBC underpads.

TABLE 7
Additional Prototypes and Samples Tested in Example 5

| | Description |
|---|---|
| Prototype 9 | Prototype 9 of Example 2 |
| Prototype 10 | Prototype 10 of Example 2 |
| Prototype 16 | Same as Prototype 9 except that the topsheet was |
| | a Spuntech 60 gsm, 35% viscose-65% PET embossed |
| | spunlace nonwoven, waffle embossing pattern, with |
| | a measured basis weight of 63.9 gsm, a thickness |
| | of 0.551 mm, and a bulk of 8.6 cm³/g |
| Prototype 17 | Same as Prototype 10, except that the topsheet was |
| | a Spuntech 60 gsm, 35% viscose-65% PET embossed |
| | spunlace nonwoven, waffle embossing pattern, with |
| | a measured basis weight of 63.9 gsm, a thickness |
| | of 0.551 mm, and a bulk of 8.6 cm³/g |
| Modified Medline | The Medline underpad with a NEN 3.0 osy PE |
| | needlepunch nonwoven |

TABLE 8
Maximum Pressure Measurements for the Example 5 Tests

| | Maximum Pressure | | | Maximum Pressure | | | Maximum Pressure | | |
|---|---|---|---|---|---|---|---|---|---|
| | (mmHg) | | | (normalized to Medline) | | | (normalized to ASBC) | | |
| | Dry | Dry | Wet | Dry | Dry | Wet | Dry | Dry | Wet |
| | Mattress | Foam | Foam | Mattress | Foam | Foam | Mattress | Foam | Foam |
| Prototype 9 | 11.3 | 16.6 | 16.2 | 88% | 68% | 84% | 85% | 95% | 93% |
| Prototype 10 | 11.7 | 16.3 | 18.4* | 91% | 67% | 95%* | 88% | 93% | 105% |
| No Underpad | 12.4 | — | — | 97% | — | — | 94% | — | — |
| ASBC | 13.2 | 17.5 | 17.4 | 104% | 71% | 90% | 100% | 100% | 100% |
| Underpad | | | | | | | | | |
| Medline | 12.8 | 24.5 | 19.3 | 100% | 100% | 100% | 97% | 140% | 111% |
| Underpad | | | | | | | | | |
| Prototype 16 | 13.3 | 20.1 | 13.0 | 104% | 82% | 67% | 100% | 115% | 75% |
| Prototype 17 | 12.3 | 20.6 | 14.0 | 96% | 84% | 72% | 93% | 118% | 80% |
| Modified | — | 16.8 | — | — | 69% | — | — | 96% | — |
| Medline | | | | | | | | | |

*Sample did not hydrate at the center of the pad when dosed and had a dry spot. Additional water was added to hydrate the underpad prior to testing.

Of all the samples tested, only Prototype 9 and Prototype 10 exhibited a lower maximum pressure than the mattress alone, where no underpad was in place. The other tested samples yielded higher maximum pressures than the “No Underpad” test. Prototype 9 and Prototype 10 also yielded lower interface pressures than the Medline underpad in a dry state on the foam pad, in a wet state on the foam pad, and in a dry state on the mattress. Prototype 16 and Prototype 17 yielded lower interface pressures than the Medline underpad in a dry state on the foam pad and in a wet state on the foam pad.

When in a dry state on the foam pad, the Modified Medline underpad exhibited a 31% reduction in maximum interface pressure compared to the Medline underpad. This reduction in TIP was not as great as those exhibited by Prototype 9 and Prototype 10, even though those samples used the same topsheet material.

### Example 6

Four prototypes of the underpad of FIGS. 2A-2C were produced and tested to assess the air permeability thereof. For each of the prototypes, the backsheet was a 76 gsm spunbond/meltblown/spunbond (SMS) nonwoven and, for each of the two laminates, the first substrate lamina was a 28 gsm, 100% viscose spunlace nonwoven, the first and second absorbent laminae each comprised 75 gsm HP500E SAP, the second substrate lamina was a 50 gsm, 50% viscose-50% PET spunlace nonwoven, and the third substrate lamina was a tissue. The prototypes had different topsheets, as set forth in TABLE 9. The Medline and ASBC underpads were tested as well.

TABLE 9
Topsheets Used for the Example 6 Prototypes

| | Topsheet |
|---|---|
| Prototype 18 | Spuntech 75 gsm, 100% PET embossed spunlace |
| | nonwoven, hills embossing pattern |
| Prototype 19 | NEN 3.0 osy PE needlepunch nonwoven |
| Prototype 20 | Shalag 50 gsm two-layered nonwoven, through-air |
| | bonded ADL disposed there between |
| Prototype 21 | 13.5 gsm spunbond nonwoven treated with a soy |
| | protein, with a measured basis weight of 13.8 gsm, a |
| | thickness of 0.124 mm, and a bulk of 9.0 cm³/g |

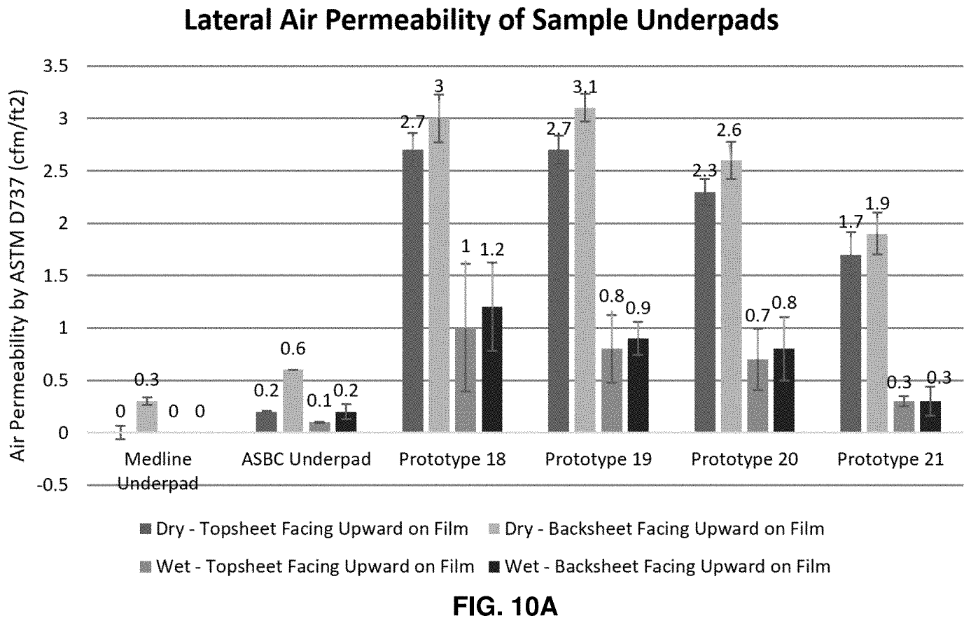

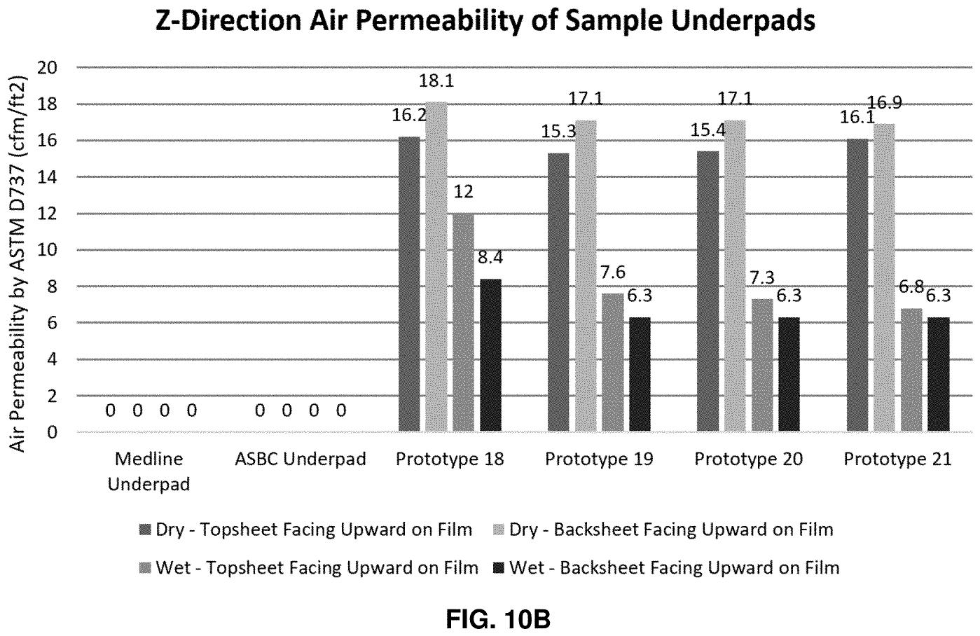

Each of the prototypes, the Medline underpad, and the ASBC underpad was tested for lateral air permeability using a TexTest FX3300 Labair IV in accordance with ASTM D737. To begin, a 250 mm×380 mm sample of each of the underpads was cut and an impermeable film was placed on top of the topsheet before clamping the sample with the FX3300 instrument. Four measurements were taken on different spots of the underpad and averaged. Thereafter, the sample was flipped such that the impermeable film was placed on top of the backsheet. The sample was again clamped with the FX3300 instrument and four measurements were taken on different spots of the underpad and averaged. After these measurements were obtained, the entire process was repeated after wetting the sample. The sample was wetted by applying a 570 ml dose of a 0.9% saline solution and permitting the sample to equilibrate before measuring the lateral air permeability. Each of the underpads was also tested for air permeability in a direction perpendicular to the inner and outer surfaces of the underpad (“z-direction air permeability”) using the above-described test procedure, except that no impermeable film was placed on the samples. FIG. 10A shows the results for the lateral air permeability test, and FIG. 10B shows the results for the z-direction air permeability test.

Each of the prototypes exhibited significantly greater lateral air permeability than the Medline and ASBC underpads, although the lateral air permeability of Prototype 21 was similar to that of the ASBC underpad when wet. The prototypes yielded similar z-direction air permeabilities.

### Example 7

A test was performed to assess the degree to which the surface of an underpad would laterally displace before shearing between the surface and a user's skin occurred. Six samples were tested: three prototypes, the Medline underpad, the ASBC underpad, and a COVIDIEN™ underpad. The tested prototypes included Prototypes 9 and 10 of Example 2 and Prototype 22, which comprised a single absorbent laminate without a topsheet or a backsheet. The laminate of Prototype 22 was the same as each of those used in Prototypes 9 and 10.

To perform the test, the sample was marked with four sets of lines: first and second sets having lines spaced 25 mm and 50 mm apart, respectively, in the cross direction (CD) and third and fourth sets spaced 25 mm and 50 mm apart, respectively, in the machine direction (MD). For each of the sets of marks, the sample was placed on a balance, a ruler was placed on the sample such that major graduation lines on the ruler aligned with the marks, and the balance was tared. Each of the tester's index fingers, with the fingernail pointed upward, was marked with a vertical line using a ballpoint pen. The tester placed both index fingers on the sample such that the lines on the left and right index fingers aligned with the left and right marks, respectively, of the set. The index fingers were pressed against the surface of the sample such that the angle between the surface and each of the index fingers was approximately 30°. The pressing was done such that the index fingers applied substantially the same force. When the balance read 750 g±50 g, the index fingers were slowly moved apart laterally while maintaining pressure against the surface to prevent slipping during the lateral movement. The distance between the two lines on the index fingers was measured with the ruler just before at least one of the fingers slipped. Displacement was calculated by subtracting the measured distance from the initial spacing.

After these measurements were taken, the test procedure was repeated after hydrating the samples. Each of the samples was hydrated using 0.003 ml of tap water per square millimeter of the surface of the sample. The water was poured in an aluminum pan and the sample was placed with its surface (e.g., the topsheet) facing down into the pan. The pan was tilted side-to-side and end-to-end until the sample absorbed the water. For each of Prototypes 9 and 10, the water was poured directly onto the topsheet of the sample in the vicinity of the markings on the sample to achieve sufficient wetting. Prototype 22 was only tested for CD displacement when hydrated.

To determine the pressure applied in each of the tests, two pieces of transparent tape were placed adjacent to each other on a piece of paper and colored with a green marker. Each of the tester's index fingers was pressed against a respective one of the colored tapes at an angle of 30° with respect to the tape surface and thereafter raised from the tape to leave an outline of the fingertip. The area of the fingertips was measured and calculated using a caliper. Pressure was calculated by dividing the applied force by the total fingertip area. The area of the tester's fingertips was 517 mm².

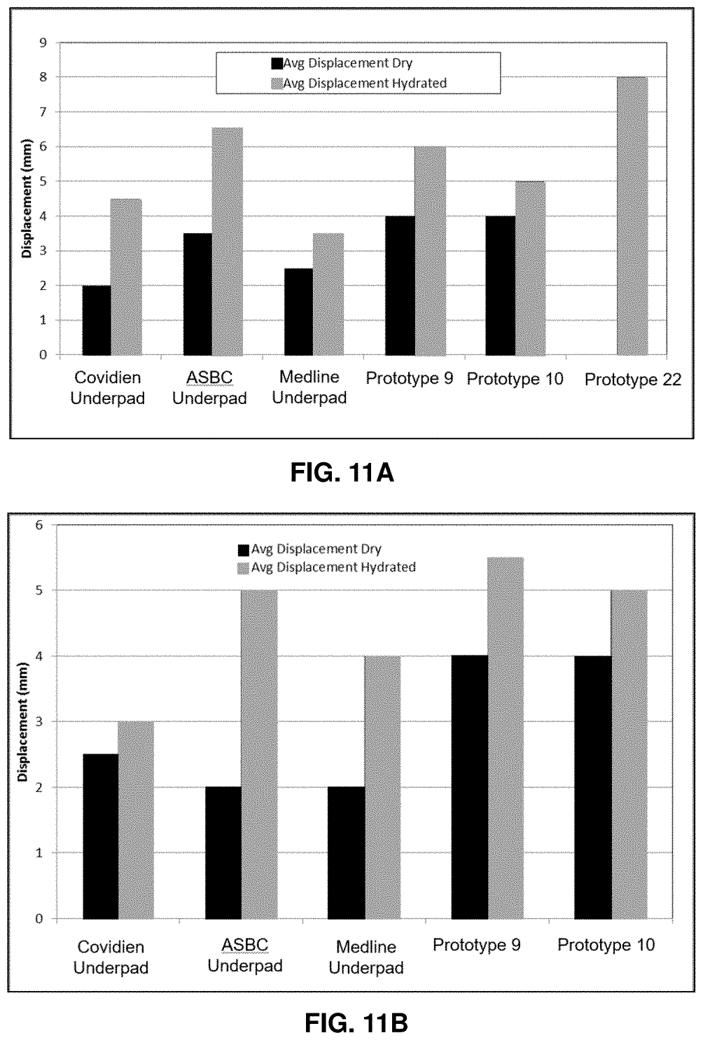

FIGS. 11A and 11B show the displacements for the CD and MD tests, respectively. In the dry state, the displacements measured for Prototypes 9 and 10 were 60% larger in the CD and 100% larger in the MD than those measured for the Medline underpad. After the samples were hydrated, the displacements measured for Prototype 9 were 71% larger in the CD and 37% larger in the MD, and those measured for Prototype 10 were 43% larger in the CD and 25% larger in the MD, compared to the Medline underpad. The applied pressure during the tests ranged between 102 and 110 mmHg.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An absorbent article comprising:
a liquid-permeable nonwoven topsheet having a bulk greater than or equal to 10 cubic centimeters per gram (cm³/g);
a backsheet; and
one or more absorbent laminates, each disposed between the topsheet and the backsheet and comprising one or more absorbent laminae and one or more substrate laminae, wherein:
each of the absorbent lamina(e) comprises superabsorbent polymer (SAP) particles and an adhesive; and
each of the substrate lamina(e) comprises a nonwoven or a tissue.

2. The absorbent article of claim 1, wherein for each of the laminate(s):
the one or more absorbent laminae comprise two or more absorbent laminae;
the SAP particles in each of the absorbent laminae have a basis weight between 20 and 130 grams per square meter (gsm); and
a first one of the substrate lamina(e) comprises a nonwoven and is disposed between first and second ones of the absorbent laminae.

3. The absorbent article of claim 2, wherein the nonwoven of the first substrate lamina comprises viscose fibers and, optionally, polyethylene terephthalate (PET) fibers.

4. The absorbent article of claim 2, wherein for each of the laminate(s):
the one or more substrate laminae comprise three or more substrate laminae;
the first absorbent lamina is disposed between the first substrate lamina and a second one of the substrate laminae; and
the second absorbent lamina is disposed between the first substrate lamina and a third one of the substrate laminae.

5. The absorbent article of claim 4, wherein at least one of the second and third substrate laminae comprises a tissue.

6. The absorbent article of claim 5, wherein for at least one of the laminate(s), the second substrate lamina is disposed on the topsheet such that the second substrate lamina is disposed closer to the topsheet than is the third substrate lamina.

7. The absorbent article of claim 6, wherein the second substrate lamina comprises a nonwoven and the third substrate lamina comprises a tissue.

8. The absorbent article of claim 7, wherein the topsheet has a basis weight between 10 and 65 gsm.

9. The absorbent article of claim 6, wherein the second substrate lamina comprises a tissue and the third substrate lamina comprises a nonwoven.

10. The absorbent article of claim 9, wherein the topsheet has a basis weight between 65 and 120 gsm.

11. The absorbent article of claim 6, wherein:
the nonwoven of the first substrate lamina has a basis weight between 20 and 40 gsm; and
for each of the second and third substrate laminae, if the substrate lamina comprises a nonwoven, the nonwoven has a basis weight between 40 and 60 gsm.

12. The absorbent article of claim 1, wherein a thickness of the topsheet is greater than or equal to 0.50 millimeters (mm).

13. The absorbent article of claim 1, wherein the backsheet comprises a nonwoven and does not comprise a liquid-impermeable film.

14. An absorbent article comprising:
a liquid-permeable topsheet comprising first and second topsheet layers;
a backsheet; and
an absorbent core disposed between the topsheet and the backsheet;
wherein:
the topsheet has a slidable region that overlies at least a majority of the absorbent core;
an adhesive bonds the first topsheet layer to the second topsheet layer and is disposed outside of the slidable region such that a portion of the first topsheet layer is slidable relative to the second topsheet layer within the slidable region; and
a coefficient of friction between the first and second topsheet layers is lower than a coefficient of friction between the first topsheet layer and the skin of a wearer.

15. The absorbent article of claim 14, wherein each of the first and second topsheet layers comprises a nonwoven and at least a portion of the first topsheet layer is gathered within the slidable region.

16. The absorbent article of claim 3, wherein for each of the laminate(s):
the one or more substrate laminae comprise three or more substrate laminae;
the first absorbent lamina is disposed between the first substrate lamina and a second one of the substrate laminae; and
the second absorbent lamina is disposed between the first substrate lamina and a third one of the substrate laminae.

17. The absorbent article of claim 7, wherein:
the nonwoven of the first substrate lamina has a basis weight between 20 and 40 gsm; and
for each of the second and third substrate laminae, if the substrate lamina comprises a nonwoven, the nonwoven has a basis weight between 40 and 60 gsm.

18. The absorbent article of claim 8, wherein:
the nonwoven of the first substrate lamina has a basis weight between 20 and 40 gsm; and
for each of the second and third substrate laminae, if the substrate lamina comprises a nonwoven, the nonwoven has a basis weight between 40 and 60 gsm.

19. The absorbent article of claim 9, wherein:
the nonwoven of the first substrate lamina has a basis weight between 20 and 40 gsm; and
for each of the second and third substrate laminae, if the substrate lamina comprises a nonwoven, the nonwoven has a basis weight between 40 and 60 gsm.

20. The absorbent article of claim 10, wherein:
the nonwoven of the first substrate lamina has a basis weight between 20 and 40 gsm; and
for each of the second and third substrate laminae, if the substrate lamina comprises a nonwoven, the nonwoven has a basis weight between 40 and 60 gsm.
